(19) United States
(12) Patent Application Publication
(10) Pub. No.: US 2019/0053739 A1
(43) Pub. Date: Feb. 21, 2019

(54) WEARABLE APPARATUS, SETTLEMENT SYSTEM, AND SETTLEMENT METHOD

(71) Applicant: SEIKO EPSON CORPORATION, Tokyo (JP)

(72) Inventors: Katsutoyo INOUE, Azumino-shi (JP); Tatsuya KITAZAWA, Ikeda-machi (JP); Hiroaki HOSOMI, Minowa-machi (JP)

(73) Assignee: SEIKO EPSON CORPORATION, Tokyo (JP)

(21) Appl. No.: 16/079,448

(22) PCT Filed: Feb. 6, 2017
(86) PCT No.: PCT/JP2017/004269

§ 371 Date: Aug. 23, 2018

(30) Foreign Application Priority Data
Feb. 24, 2016 (JP) 2016-032760
May 2, 2016 (JP) 2016-092314
May 13, 2016 (JP) 2016-096809

(51) Int. Cl. A61B 5/1171; A61B 5/0205; A61B 5/00; G06Q 40/02; G06F 1/16; G06F 21/32

(52) U.S. Cl. CPC A61B 5/1171; A61B 5/02055; A61B 5/6843; A61B 5/02433; G06Q 40/02; G06F 1/163; G06F 21/32; A61B 5/7221

(57) ABSTRACT

A wearable apparatus which rapidly outputs a result of personal authentication without performing a personal authentication process on a user every time. A wearable apparatus includes an authentication processing unit that performs personal authentication for a user wearing the wearable apparatus, a detection unit that detects a mounting state of the wearable apparatus, a determination processing unit that determines whether a result of the personal authentication is valid or invalid on the basis of the mounting state detected by the detection unit, and a control unit that omits the personal authentication in the authentication processing unit, and outputs a result of the personal authentication in a case where the user is personally authenticated by the authentication processing unit, and then the result of the personal authentication is determined as being valid by the determination processing unit.

### TECHNICAL FIELD

The present invention relates to a wearable apparatus, a settlement system, and a settlement method.

### BACKGROUND ART

In the related art, as disclosed in PTL 1, there is a wearable apparatus which is mounted on a user's body such as the wrist, and performs personal authentication by using biological information of the user. In the wearable apparatus, a result of the personal authentication is transmitted to an external apparatus, and the user subjected to the personal authentication can operate the external apparatus.

### CITATION LIST

### Patent Literature

PTL 1: JP-A-2006-48667

### SUMMARY OF INVENTION

### Technical Problem

However, in the wearable apparatus disclosed in PTL 1, a user authentication process is performed whenever personal authentication is necessary, and thus an external apparatus is required to wait for the time until a collation result of the personal authentication is completed. In PTL 1, as an authentication method, collation of a vein pattern is used, but, for example, in a case where a vein imaging position is adjusted again, the time required for an authentication process is not constant depending on the user's operation time.

Examples of cases of outputting card information of a credit card or a debit card requiring personal authentication to an external apparatus may include payment of a toll at an exit of a toll road and payment of a railroad fare or a bus fare. In these cases, a payment process is required to be rapidly performed, but, in the wearable apparatus disclosed in PTL 1, the personal authentication process requires a considerable time, and the time required for the personal authentication process is not constant, so that it is difficult for the wearable apparatus to be used for such an application.

With respect to the wearable apparatus disclosed in PTL 1, despite handling of information requiring high security, such as card information, in a case where the wearable apparatus is unmounted contrary to a user's intention, a function of preventing illegal use of the wearable apparatus is not taken into consideration.

In a wearable apparatus of the related art, in a case where a result of personal authentication for a user is desired to be used by reliable another user, for example, in a case where family members share a single wearable apparatus, a wife is desired to perform a financial process on behalf of her husband or a case where a guardian or the like is desired to perform a process related to property on behalf of a ward, the result of personal authentication for the user cannot be used by another user. Thus, it is desirable to provide a wearable apparatus enabling a result of personal authentication for a user to be easily and safely trusted (taken over) to a reliable substitute.

### Solution to Problem

The present invention has been made in order to solve at least some of the above-described problems, and provides a wearable apparatus which outputs a result of personal authentication without performing a personal authentication process on a user again for a period in which the result of personal authentication is effective as information.

The present invention provides a wearable apparatus which enables a result of personal authentication for a user performed by the wearable apparatus to be safely taken over to a substitute through a simple process.

### Application Example 1

A wearable apparatus according to this application example is configured to be worn by a user, and includes an authentication unit that performs personal authentication for the user wearing the wearable apparatus; a detection unit that detects a mounting state of the wearable apparatus; a determination unit that determines whether a result of the personal authentication is valid or invalid on the basis of the mounting state detected by the detection unit; and a control unit that omits the personal authentication in the authentication unit, and outputs a result of the personal authentication, in a case where the user is personally authenticated by the authentication unit, and then the result of the personal authentication is determined as being valid by the determination unit.

According to this application example, the wearable apparatus determines whether a result of personal authentication is valid or invalid on the basis of a mounting state after a user is personally authenticated, and outputs a determination result. For example, in a period in which a mounting state is maintained after the user is personally authenticated, it is output that a result of personal authentication is valid. Personal authentication is not performed every time during the period.

Therefore, in a period in which a result of personal authentication is valid, the result of personal authentication can be output without performing a personal authentication process on a user again.

### Application Example 2

The wearable apparatus further includes a reception unit that receives an unmounting instruction for the wearable apparatus; and a notification unit that performs a notification of information by stimulating senses of a living body including at least one of a vision, an auditory sense, and a tactile sense, and the control unit causes the notification unit to perform a notification of abnormality information in a case where it is detected by the detection unit that the wearable apparatus is unmounted during non-reception in which the user does not give the unmounting instruction to the reception unit.

According to this application example, in a case where the wearable apparatus is unmounted during non-reception of the unmounting instruction, a notification of abnormality information is sent.

Therefore, the wearable apparatus has a function of preventing illegal use of the wearable apparatus in a case where the wearable apparatus is unmounted contrary to the user's intention.

### Application Example 3

The wearable apparatus further includes a storage unit that stores financial process information which enables a financial process using at least one of a credit card function, a cash card function, a debit card function, and a net banking function to be performed, the control unit performs the financial process in a case where the user wearing the wearable apparatus is personally authenticated by the authentication unit, and then the result of the personal authentication is determined as being valid by the determination unit, and the control unit causes the financial process not to be performed in a case where the result of the personal authentication is determined as being invalid.

According to this application example, since a wearable apparatus subjected to personal authentication can perform a financial process, and a wearable apparatus not subjected to personal authentication cannot perform a financial process, it is possible to prevent spoofing of a user.

### Application Example 4

The wearable apparatus further includes a mounting unit that is used for the wearable apparatus to be worn by the user, the mounting unit forms an annular shape around the wrist of the user, the mounting unit has an opening/closing portion which opens and closes the annular shape, and, in a case where the opening/closing portion is opened, the detection unit detects that the wearable apparatus is unmounted.

According to this application example, the mounting unit may be, for example, a belt used for the wearable apparatus to be mounted on the wrist, and the opening/closing portion may employ a pinback type buckle which attaches the belt to the arm.

### Application Example 5

The wearable apparatus further includes a mounting unit that is used for the wearable apparatus to be worn by the user, the mounting unit forms an annular shape around the wrist of the user, the mounting unit has an adjustment portion which variably adjusts a circumferential length of the annular shape, and, in a case where a circumferential length in a mounting state is increased by a predetermined length by the adjustment portion, the detection unit detects that the wearable apparatus is unmounted.

According to this application example, the mounting unit may be, for example, a bracelet used for the wearable apparatus to be mounted on the wrist, and the adjustment portion may employ, for example, a one-side opening type buckle, a both-hole type buckle, or a double lock type buckle which varies a circumferential length of the annular shape and is mounted on the wrist.

### Application Example 6

The authentication unit acquires biological information of the user, and performs the personal authentication.

According to this application example, it is possible to identify an individual by using a physical feature of a user on the basis of biological information.

### Application Example 7

The authentication unit performs the personal authentication by using a password entered by the user.

According to this application example, it is possible to identify an individual by using a password memorized by a user.

### Application Example 8

The authentication unit is connected to an authentication server via a computer communication network, and performs the personal authentication by collating information acquired from the user with specific information of the user stored in the authentication server.

According to this application example, it is possible to perform personal authentication on the basis of specific information stored in the authentication server, and thus to increase, for example, accuracy of personal authentication.

### Application Example 9

The wearable apparatus further includes a biological reaction information acquisition unit that acquires biological reaction information by detecting a biological reaction of the user, and the determination unit determines whether the result of the personal authentication is valid or invalid on the basis of information regarding whether or not the biological reaction information is continuously measured by the biological reaction information acquisition unit.

According to this application example, a result of personal authentication can be maintained to be valid while the biological reaction information is continuously measured. In other words, in a case where there is no special change in biological reaction information, a result of personal authentication is determined as being valid, and thus personal authentication is not performed every time.

### Application Example 10

The wearable apparatus further includes at least one of a clocking unit that measures a time point, a position information acquisition unit that acquires a position of the wearable apparatus, and a biological reaction information acquisition unit that acquires biological reaction information by detecting a biological reaction of the user, and, in at least one of a case where the time point becomes a predetermined time point, a case where the position is deviated from a predetermined range, and a case where the biological reaction information changes, the authentication unit performs the personal authentication for the user.

According to this application example, in a case where biological reaction information is not continuously measured, personal authentication may be performed to be confirmed again. In a case where a predetermined time point comes or a position of the wearable apparatus is deviated from a predetermined range, personal authentication may be performed. Even in a case where mounting is continuously performed, personal authentication is performed at the above-described timing, and thus, for example, it is possible to prevent illegal use.

### Application Example 11

The wearable apparatus further includes a temporary reception unit that receives temporary unmounting of the wearable apparatus, and, in a case where the temporary unmounting is received from the personally authenticated user, the control unit causes the authentication unit to perform simple personal authentication.

According to this application example, in a case where a personally authenticated user desires to temporarily wear the wearable apparatus again, personal authentication can be performed through a simple operation.

### Application Example 12

The detection unit measures contact pressing against the wrist of the user, and detects the mounting state on the basis of the contact pressing of when the user wears the wearable apparatus.

According to this application example, a user can detect that the wearable apparatus is worn on the basis of contact pressing in which a feature of the wrist of the user is reflected.

### Application Example 13

The wearable apparatus further includes a power generation unit that generates power, and the wearable apparatus is operated by using power from the power generation unit.

According to this application example, the wearable apparatus includes the power generation unit generating power, and can thus improve normal operability in a mounting state.

### Application Example 14

A wearable apparatus according to this application example is configured to be worn by a first user and a second user, and includes an authentication unit that performs personal authentication for the first user and the second user wearing the wearable apparatus; a detection unit that detects a mounting state of the wearable apparatus; a determination processing unit that performs information processing required to determine whether a result of the personal authentication is valid or invalid on the basis of the mounting state detected by the detection unit; a control unit that outputs information based on a result of the personal authentication in the authentication unit; and a inheritance reception unit that receives a right inheritance instruction for the wearable apparatus.

According to this application example, in the wearable apparatus for which personal authentication for the first user and the second user is performed, the right can be taken over between the first user and the second user by the inheritance reception unit. For example, information based on a result of personal authentication for the first user (user) performed for the wearable apparatus can be used by the second user (substitute) through a simple process of receiving a right inheritance instruction from the first user. In this case, personal authentication for the second user is performed, and thus it is possible to increase the safety.

Therefore, it is possible to provide the wearable apparatus which can safely inherit a result of personal authentication for the first user (user) performed by the wearable apparatus to the second user (substitute) according to a simple process.

### Application Example 15

In the wearable apparatus according to Application Example 14, the wearable apparatus further includes a notification unit that performs a notification of information by stimulating senses of a living body including at least one of a vision, an auditory sense, and a tactile sense, and the control unit causes the notification unit to perform a notification of non-inheritance information in a case where the second user is personally authenticated by the authentication unit during non-inheritance in which the inheritance reception unit does not receive the right inheritance instruction from the first user.

According to this application example, the second user wearing the wearable apparatus can acquire non-inheritance information indicating that the right is not taken over from the first user from the notification unit.

### Application Example 16

In the wearable apparatus according to Application Example 14 or 15, the wearable apparatus further includes a storage unit that stores first user information regarding the first user, and the control unit outputs the first user information regarding the first user in a case where the second user is personally authenticated by the authentication unit, and a result of the personal authentication for the second user is determined as being valid by the determination processing unit during inheritance in which the inheritance reception unit receives the right inheritance instruction from the first user to the second user.

According to this application example, the wearable apparatus having performed personal authentication for the second user can output the first user information during inheritance in which the right inheritance instruction from the first user is received.

### Application Example 17

In the wearable apparatus according to Application Example 16, the first user information includes at least one of my number information, credit card information, cash card information, debit card information, and net banking information of the first user.

According to this application example, the first user information may include a personal identification number or financial information of the first user.

### Application Example 18

In the wearable apparatus according to any one of Application Examples 14 to 17, the authentication unit acquires biological information of users including the first user and the second user, and performs the personal authentication.

According to this application example, it is possible to identify an individual by using a physical feature of a user on the basis of biological information.

### Application Example 19

In the wearable apparatus according to Application Example 18, the authentication unit performs the personal authentication by using a password entered by the user.

According to this application example, it is possible to identify an individual by using a password memorized by a user.

### Application Example 20

In the wearable apparatus according to Application Example 18 or 19, the authentication unit is connected to an authentication server via a computer communication network, and performs the personal authentication by collating information acquired from the user with specific information of the user stored in the authentication server.

According to this application example, it is possible to perform personal authentication on the basis of specific information stored in the authentication server, and thus to increase, for example, accuracy of personal authentication.

### Application Example 21

In the wearable apparatus according to any one of Application Examples 18 to 20, the wearable apparatus further includes a biological reaction information acquisition unit that acquires biological reaction information by detecting a biological reaction of the user, and the determination processing unit determines whether the result of the personal authentication is valid or invalid on the basis of information regarding whether or not the biological reaction information is continuously measured by the biological reaction information acquisition unit.

According to this application example, a result of personal authentication can be maintained to be valid while the biological reaction information is continuously measured. In other words, in a case where there is no special change in biological reaction information, a result of personal authentication is determined as being valid, and thus personal authentication is not performed every time.

### Application Example 22

In the wearable apparatus according to any one of Application Examples 18 to 21, the wearable apparatus further includes at least one of a clocking unit that measures a time point, a position information acquisition unit that acquires a position of the wearable apparatus, and a biological reaction information acquisition unit that acquires biological reaction information by detecting a biological reaction of the user, and, in at least one of a case where the time point becomes a predetermined time point, a case where the position is deviated from a predetermined range, and a case where the biological reaction information changes, the authentication unit performs the personal authentication for the user.

According to this application example, in a case where biological reaction information is not continuously measured, personal authentication may be performed to be confirmed again. In a case where a predetermined time point comes or a position of the wearable apparatus is deviated from a predetermined range, personal authentication may be performed. Even in a case where mounting is continuously performed, personal authentication is performed at the above-described timing, and thus, for example, it is possible to prevent illegal use.

### Application Example 23

In the wearable apparatus according to any one of Application Examples 14 to 22, the wearable apparatus further includes a power generation unit that generates power, and the wearable apparatus is operated by using power from the power generation unit.

According to this application example, the wearable apparatus includes the power generation unit generating power, and can thus improve normal operability in a mounting state.

### Application Example 24

A settlement system according to this application example employs the wearable apparatus described above.

According to this application example, in the settlement system, in the wearable apparatus for which personal authentication for the first user and the second user is performed, the right can be taken over between the first user and the second user. For example, a payment process using financial information owned by the first user who is personally authenticated by the wearable apparatus can be performed on behalf thereof by the second user who takes over the financial information by using the wearable apparatus and is personally authenticated.

### Application Example 25

A settlement method according to this application example employs a wearable apparatus which is configured to be worn by a first user and a second user and to perform personal authentication for the first user and the second user, including causing the first user to wear the wearable apparatus so as to perform the personal authentication, causing the first user to inherit the right of the wearable apparatus to the second user, and causing the second user to wear the wearable apparatus so as to perform the personal authentication, so that the second user can perform settlement by using the wearable apparatus on behalf of the first user.

According to this application example, it is possible to provide the settlement method in which the second user can perform settlement by using the wearable apparatus on behalf of the first user.

### BRIEF DESCRIPTION OF DRAWINGS

FIG. 1 is an explanatory diagram illustrating an overview of a usage form of a wearable apparatus.

FIG. 2 is a diagram illustrating an overview of an authentication sequence.

FIG. 3 is a block diagram illustrating a schematic configuration of the wearable apparatus.

FIG. 4 is a diagram illustrating an example of a personal information table.

FIG. 5 is a diagram illustrating an example of registered authentication data.

FIG. 6 is a diagram illustrating an example of result data.

FIG. 7 is a flowchart illustrating a flow of a main control process.

FIG. 8 is a flowchart illustrating a flow of an initial registration process.

FIG. 9 is a flowchart illustrating a flow of a mounting event process.

FIG. 10 is a flowchart illustrating a flow of an unmounting event process.

FIG. 11 is a flowchart illustrating a flow of an authentication request process.

FIG. 12 is a flowchart illustrating a flow of an unmounting event process.

FIG. 13 is an explanatory diagram illustrating an overview of a usage form of a wearable apparatus.

FIG. 14 is an explanatory diagram illustrating an overview of an authentication sequence.

FIG. 15 is a block diagram illustrating a schematic configuration of the wearable apparatus.

FIG. 16 is a diagram illustrating an example of a personal information table.

FIG. 17 is a diagram illustrating an example of result data.

FIG. 18 is a flowchart illustrating a flow of a main control process.

FIG. 19 is a flowchart illustrating a flow of a mounting event process.

FIG. 20 is a flowchart illustrating a flow of an authentication request process.

FIG. 21 is a flowchart illustrating a flow of a inheritance event process.

FIG. 22 is a diagram illustrating an example of a inheritance destination designation screen.

FIG. 23 is a diagram illustrating an example of a inheritance information designation screen.

FIG. 24 is an explanatory diagram illustrating an overview of a usage form of a wearable apparatus according to a modification example.

FIG. 25 is an explanatory diagram illustrating an overview of a usage form of a wearable apparatus according to another modification example.

FIG. 26 is an explanatory diagram illustrating an overview of a usage form of a wearable apparatus according to still another modification example.

FIG. 27 is an explanatory diagram illustrating an overview of a usage form of a wearable apparatus according to still another modification example.

FIG. 28 is an explanatory diagram illustrating an overview of a usage form of a wearable apparatus according to still another modification example.

### DESCRIPTION OF EMBODIMENTS

Hereinafter, embodiments of the present invention will be described with reference to the drawings. In the following diagrams, a scale of each unit or each screen is different from actual one since each unit or each screen is exaggerated to be able to be recognized.

### Embodiment 1

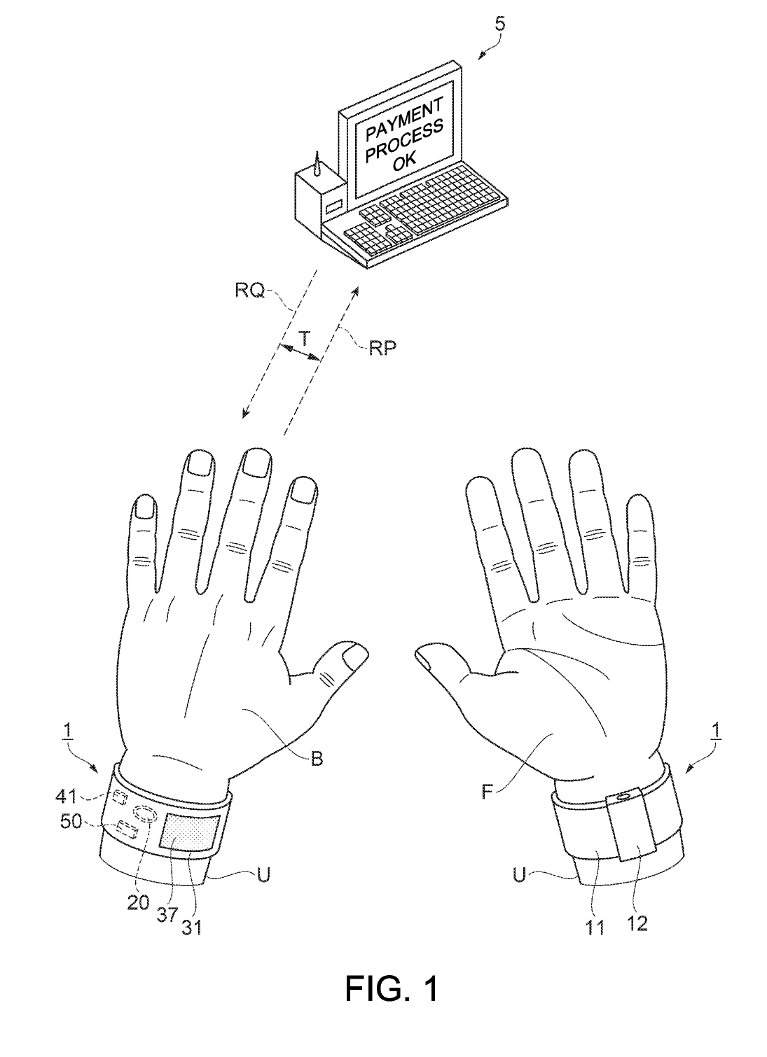

FIG. 1 is an explanatory diagram illustrating an overview of a usage form of a wearable apparatus.

A wearable apparatus 1 is a terminal mounted on the wrist of a user U as a user. In the wearable apparatus 1, a display portion 31 of a flexible type of being able to be flexibly deformed which performs various displays and an operation unit 37 of a touch panel type covering the display portion 31 are incorporated into a band 11 wound on the circumference of the wrist. The wearable apparatus 1 illustrated in FIG. 1 is mounted on the left wrist of the user U, includes the display portion 31 and the operation unit 37 on the back of the hand B side, and includes an opening/closing portion 12 attaching and fixing the band 11 to the wrist on the flat of the hand F side of the same left hand. A control unit 50 which is a central processing unit (CPU), a biological information measurement unit 20 which measures biological information of the user U, a communication unit 41 which transmits and receives various pieces of data through wireless communication, and the like are built into the band 11 along with associated circuits.

A charge system 5 performs a payment process (corresponding to a financial process) of the charge for a service used by the user U on the basis of financial process information enabling a financial process to be performed, such as card information of a cash card, a credit card, a debit card, or the like, or account information in net banking or the like. The charge system 5 is installed at a location where the charge is paid, and transmits, for example, a command RQ for requesting card information to the wearable apparatus 1 through wireless communication if the user U comes close thereto. In a case where the command RQ is received, the wearable apparatus 1 transmits a command RP including the card information to the charge system 5 after time T elapses.

In the wearable apparatus 1, card information of the user U is stored in a storage unit 70 (which will be described later) The wearable apparatus 1 is required to determine whether or not the card information can be transmitted to the charge system 5. In other words, it is necessary to confirm that a person wearing the wearable apparatus 1 is the user U (the user U having used a service) and then to transmit the card information of the user U. To do so, a personal authentication process on the user U is performed.

In a case where card information is requested by the charge system 5, the wearable apparatus 1 is required to rapidly transmit the card information.

For example, in a case where the charge system 5 is an electronic toll collection (ETC) system provided at an exit to which a toll of a vehicle is paid, it is necessary to transmit card information within a predetermined time without stopping a traveling car and to finish a toll payment process. In this case, the wearable apparatus 1 is used instead of an existing ETC card and an on-vehicle device into which the ETC card is inserted. In other words, transmission and reception of card information or the like using the wearable apparatus 1 are required to be performed within at least a predetermined time for which the existing on-vehicle device and ETC card perform transmission and reception.

However, in a personal authentication process on the user U, the authentication process requires a considerable time. For example, in the method using vein pattern identification disclosed in PTL 1, capturing of a vein image and a pattern analysis process are performed. In the processes, it takes time to perform the imaging process and the pattern analysis process. If there is a technique of being capable of performing the processes at a high speed, in a case where a mounting position of the wearable apparatus is required to be readjusted by the user U during a personal authentication process, the time required for the process is not predicted and thus is not constant.

The wearable apparatus 1 can confirm that a person wearing the wearable apparatus 1 is the user U (can obtain a result of the user U being personally authenticated) within a predetermined time and can transmit card information or the like of the user U, without depending on the time required for a personal authentication process on the user U or the time required for an operation or the like performed by the user U during a personal authentication process.

Next, an overview of a realization method thereof will be described.

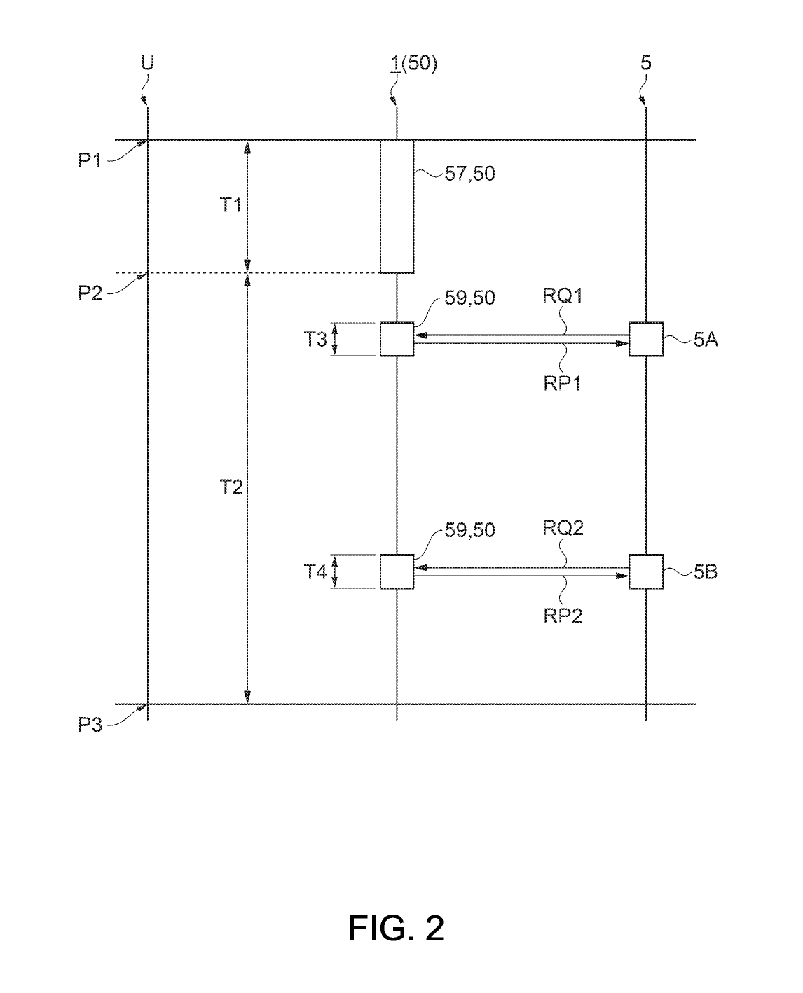

FIG. 2 is a diagram illustrating an overview of an authentication sequence.

A sequence diagram of FIG. 2 illustrates mutually cooperating portions according to a flow of time (from the top to the bottom) in an operation (U) performed by the user U, a process in the control unit 50 of the wearable apparatus 1, and a process in the charge system 5. As will be described later in detail, the control unit 50 has an authentication processing unit 57 and a determination processing unit 59 as functional units.

If the user U wears the wearable apparatus 1 (operation P1), the wearable apparatus 1 performs a personal authentication process on the user U (authentication processing unit 57). In the personal authentication process, a vein pattern of the user U registered in the wearable apparatus 1 in advance is collated with a vein pattern analyzed from a vein captured image generated by controlling an imaging sensor 21.

Here, the time required for the personal authentication process is set to a time T1. A time period after the personal authentication process is finished (operation P2) until the user U removes and thus does not wear the wearable apparatus 1 (operation P3) is set to a time T2.

For the time T2, if a payment process 5A occurs in the charge system 5, a command RQ1 for requesting card information for the payment process is transmitted from the charge system 5 to the wearable apparatus 1. In the wearable apparatus 1, the determination processing unit 59 and the control unit 50 perform a process corresponding to the command RQ1, and transmit (response about) a command RP1 including the card information of the user U. In this case, the control unit 50 determines that a result of the personal authentication process is valid without performing a personal authentication process in the authentication processing unit 57, so as to confirm that a person wearing the wearable apparatus 1 is the user U, and then transmits the card information of the user U to the charge system 5 side. A process time until a response about the command RP1 is made from reception of the command RQ1 in the control unit 50 is set to a time T3.

If another payment process 5B occurs in the charge system 5 for the time T2, a command RQ2 for requesting card information is transmitted to the wearable apparatus 1. In the control unit 50 of the wearable apparatus 1, the determination processing unit 59 transmits (responses about) a command RP2 including the card information of the user U through the above-described same process. A process time until a response about the command RP2 is made from reception of the command RQ2 in the control unit 50 is set to a time T4. The payment process 5B is a process in the charge system 5, but may be a process in a separate system which is different from the charge system 5.

Here, the time required for each process will be focused. The time T1 is the time required for a personal authentication process, and includes a process time for performing the imaging process and the pattern analysis process in the above-described examples. In a case where vein patterns are not collated with each other, a cause thereof may be the influence of deviation in an imaging position (vein position), and thus coping such as making the user U adjust a mounting position of the wearable apparatus 1 is necessary. Therefore, the time T1 requires at least an unfixed time in addition to a predetermined time required for analysis of a personal authentication process. In the related art, in a case where there is a request for card information or the like from the charge system 5, the time T1 is necessary in order to make a response thereto every time.

In contrast, the determination processing unit 59 which performs a process in a time period of the time T2 can perform determination through a simple step of reading the content of a flag storing whether a result of a personal authentication process is valid or invalid and determining data of the flag, and a process step is not inconstant and fixed (details thereof will be described later). In other words, each of the time T3 and the time T4 is a time shorter than the time T1, and is a fixed constant time. If the charge system 5 side waits for responses to the transmitted commands RQ1 and RQ2 for the constant time, the commands RP1 and RP2 can be acquired.

Hereinafter, a configuration of the wearable apparatus 1 which can achieve such effects will not be described in detail.

### (Configuration of Wearable Apparatus)

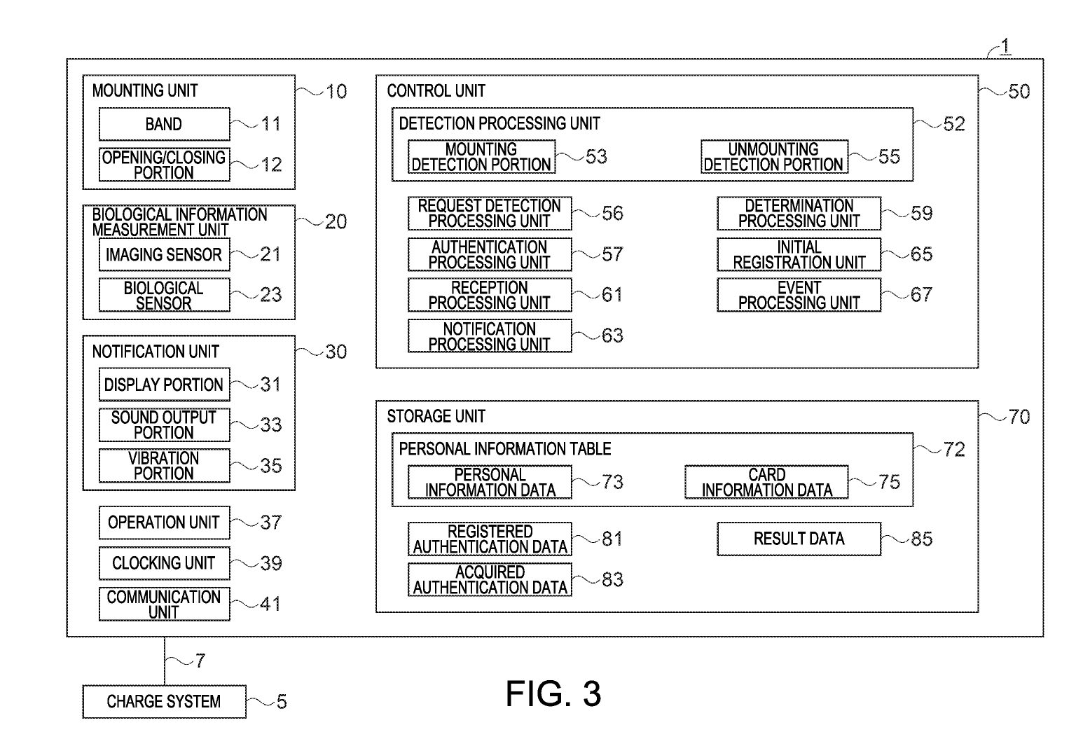

FIG. 3 is a block diagram illustrating a schematic configuration of the wearable apparatus.

The wearable apparatus 1 is configured to include a mounting unit 10, the biological information measurement unit 20, a notification unit 30, the operation unit 37, a clocking unit 39, the communication unit 41, the control unit 50, and the storage unit 70. The wearable apparatus 1 is connected to the charge system 5 via communication 7.

The mounting unit 10 is configured to include the band 11 and the opening/closing portion 12.

The band 11 and the opening/closing portion 12 are members annularly winding the wearable apparatus 1 on the circumference of the user's wrist and attaching the wearable apparatus to the wrist.

The opening/closing portion 12 is a buckle provided with a switch sensor. The buckle may employ any type such as a one-side opening type, a both-hole type, a double lock type, and a pinback type. The switch sensor is provided in an opening/closing operation portion of the buckle. In a state in which the band 11 is attached to the wrist, the switch is turned on, and an ON signal is output from the opening/closing portion 12. If the band 11 is detached from the wrist, an OFF signal is output from the opening/closing portion 12. The ON/OFF signal output from the opening/closing portion 12 is output to the control unit 50.

The switch sensor may be disposed at any position in the band 11 on the wrist side in a state in which the band 11 is wound on the wrist. In this configuration, the switch is turned on due to pressing from the wrist in a state in which the band is mounted on the wrist, and the switch is turned off if the band is detached from the wrist. A pressure sensor which measures a pressing pressure value may be used instead of the switch sensor. The pressure sensor can output pressure data in addition to an ON/OFF signal.

The biological information measurement unit 20 is configured to include the imaging sensor 21 and a biological sensor 23.

The imaging sensor 21 is a sensor including a light source and a charge coupled device (CCD), and the light source and the CCD are disposed on the wrist side of the user in a state in which the wearable apparatus 1 is mounted on the wrist of the user. Irradiation light from the light source is applied to the user from, for example, a light emitting diode (LED) light source emitting near infrared rays. The CCD performs photoelectric conversion on an amount of light reflected from blood vessels covering a human body, so as to generate an imaging signal (image signal). An image signal of a vein is extracted from the generated image by using a characteristic that reduced hemoglobin in vein blood easily absorbs near infrared rays. The extracted image signal of a vein is output to the control unit 50. As the imaging sensor 21, a CMOS sensor may be used.

The biological sensor 23 includes sensors such as a pulse wave sensor and a temperature sensor, and the sensors are all disposed on the wrist side of the user, detects a biological reaction such as a pulse waveform or a skin temperature of the user, and output biological reaction information.

The pulse wave sensor includes a light emitting element such as an LED and a light receiving element such as a photodiode. Light applied by the light emitting element is reflected at blood vessels of the wrist of the user, and reflected light is collected and is received by the light receiving element. In this case, the pulse wave sensor detects a pulse waveform of the user on the basis of change information for an amount of received light by using a phenomenon in which light reflectance differs between the expansion and the contraction of a blood vessel. Data regarding the detected pulse waveform is output to the control unit 50.

The temperature sensor is a well-known sensor which measures the skin temperature or the wrist or the temperature of a subcutaneous deep part in a contact or noncontact manner. A signal of the measured temperature is output to the control unit 50.

The notification unit 30 is configured to include the display portion 31, a sound output portion 33, and a vibration portion 35.

The display portion 31 is a display device which can display text or icons, and is, for example, a flexible type dot matrix electrophoretic display (EPD) which can be flexibly deformed as described above. Various pieces of information are displayed on the basis of a display signal which is input from the control unit 50.

The sound output portion 33 is configured to include a piezoelectric buzzer, a piezoelectric vibrator, or a piezoelectric speaker, and is a sound output device which performs various notifications based on output signals from the control unit 50.

The vibration portion 35 is a piezoelectric vibrator or a micro-vibration motor, and causes the wearable apparatus 1 to vibrate as a result of being driven on the basis of a signal which is input from the control unit 50.

The operation unit 37 is an input device configured to include a touch sensor or a touch panel covering a display surface of the display portion 31, detects a touched part according to an electrostatic capacitance method or a resistive film method, and outputs a detection signal to the control unit 50. A configuration of the operation unit 37 is not limited thereto, and may be a configuration in which a plurality of input operations can be performed. There may be a configuration in which an acceleration sensor or the like is provided to acquire an acceleration signal, an operation performed by the user is analyzed for each generation pattern of the acceleration signal, and an operation signal is output to the control unit 50. According to this configuration, for example, it is possible to realize an operation in which, in a case where the user taps a part of the wearable apparatus 1 three times, acceleration signals thereof are detected, and a mode of receiving the payment process 5A or the like and a mode of not receiving the payment process are switched.

The clocking unit 39 is a real time clock, and has clocking functions such as a timer function, a calendar function, and a clock function. Clocking data such as the date and time, a time, or the like measured by the clocking unit 39 is read by the control unit 50.

The communication unit 41 is a short-range radio adaptor for reducing power consumption as a preferred example, and is connected to various apparatuses such as a terminal such as the charge system 5, a cash register terminal, an automated teller machine (ATM), various charge payment machines, a personal computer (PC), and a smart phone (multi-function mobile phone) so as to perform communication of various pieces of data. The short-range radio adaptor may employ the same method as a method employed by a system which requests a payment process or the like, and may be, for example, a Bluetooth (registered trademark) adaptor, and may be an active Dedicated Short-Range Communication (DSRC) adaptor of 5.8 GHz band if a system which requests a payment process is, for example, an ETC system. In a case where the communication unit 41 receives various pieces of data from an external apparatus such as the charge system 5, the received data is output to the control unit 50. For example, in a case where the command RQ for requesting card information is received from the charge system 5, the command RQ is output to the control unit 50. In a case where the command RP transmitted from the control unit 50 is input, the command RP is transmitted to the charge system 5.

The communication unit 41 may have an Internet Protocol (IP) as a public wireless LAN adaptor, and may be communicably connected to various servers or other Internet of Things (IoT) apparatuses via a network such as the Internet or an intranet as a computer communication network.

The communication unit 41 performs communication with an external apparatus by using a common protocol, and a well-known encryption method is applied to the protocol, and thus security of data which is transmitted and received is ensured.

### (Control Unit)

The control unit 50 is a control device and a calculation device which are configured to include a processor such as a CPU or a digital signal processor (DSP), and integrally control the respective units of the wearable apparatus 1. The control unit 50 realizes various functions of the wearable apparatus 1 according to various programs including a control program (not illustrated) stored in the storage unit 70.

The control unit 50 has a detection processing unit 52, a request detection processing unit 56, the authentication processing unit 57, the determination processing unit 59, a reception processing unit 61, and a notification processing unit 63 as principal functional units, and integrally controls the functional units with an event processing unit 67 so as to realize the functions of the wearable apparatus 1. The control unit 50 realizes a function of an initial registration unit 65 before the functional units are realized.

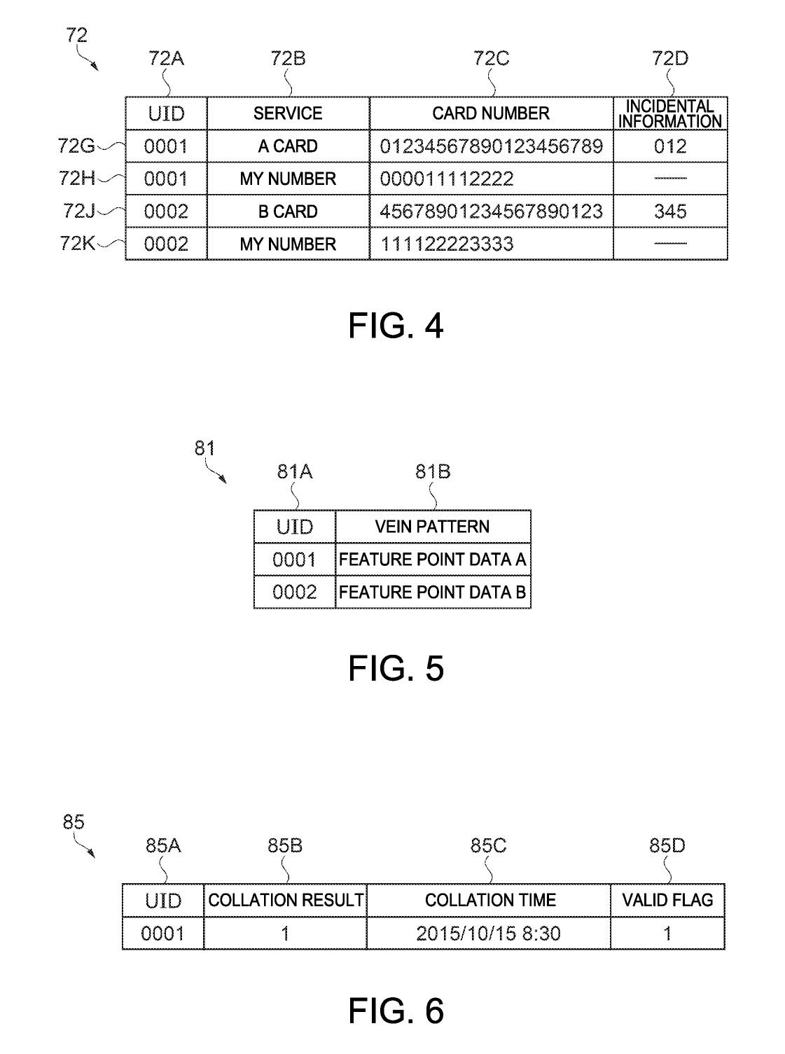

The initial registration unit 65 performs initial registration of a user using the wearable apparatus 1. Specifically, the initial registration unit 65 controls the operation unit 37 and the display portion 31 so as to acquire information such as personal information or card information of the user. The acquired information is stored in the storage unit 70 as a personal information table 72 (FIG. 4). When the personal information table is stored, a user identification (UID) for identifying the user in the control unit 50 is generated and is managed as information regarding the user.

Here, with reference to FIG. 4, the personal information table 72 will be described. FIG. 4 is a diagram illustrating an example of a personal information table. A column 72A, a column 72B, a column 72C, a column 72D, a row 72G, a row 72H, a row 72J, and a row 72K are shown in the personal information table 72. A UID is stored in the column 72A. The type of service is stored in the column 72B, a card number is stored in the column 72C, and incidental information is stored in the column 72D. The row 72G shows that a credit card service of a user having a UID of “0001” is “A card”, a card number thereof is “01234567890123456789”, and incidental information is “012”. The row 72H shows that the type of personal information of the user “0001” who is the same as in the row 72G is “my number”, and a number thereof is “000011112222”. Information regarding a user having a UID of “0002” is stored in the row 72J and the row 72K in the same form as that of “0001”.

The personal information is information such as my number, a name, an age, and an address, and may be transmitted in a case of being requested from the charge system 5 or other systems. The card information is, for example, a credit card number, a debit card number, or a cash card number, and a security code or a password is stored as incidental information as necessary.

The initial registration unit 65 controls the operation unit 37 and the display portion 31 so as to prompt a user to wear the wearable apparatus 1, and measures biological information for specifying the user individual after the user wears the wearable apparatus 1. The biological information is a vein image signal with high individual identification accuracy as a preferred example. The initial registration unit 65 controls the biological information measurement unit 20 so as to acquire a vein image signal. Well-known processes such as a binarization process, a thinning process, and feature point extraction of an image are applied to the acquired vein image signal, and thus feature point data of a vein pattern is calculated. The feature point data includes a plurality of pieces of feature point data such as a gap between or the number of branch points of the vein pattern, a length and a shape between branch points, and an angle of an included angle at a branch point. If the feature point data of the user is calculated, the initial registration unit 65 stores the feature point data in the storage unit 70 as registered authentication data 81 (FIG. 5) in correlation with a UID of the user.

Here, with reference to FIG. 5, the registered authentication data 81 will be described. FIG. 5 is a diagram illustrating an example of registered authentication data. A column 81A and a column 81B are shown in the registered authentication data 81. A UID is stored in the column 81A. Feature point data of a vein pattern is stored in the column 81B. Feature point data having a UID of “0001” is stored in the first row of the registered authentication data 81 as “feature point data A”. The feature point data A is a numerical value data group indicating feature points. There may be a plurality of numerical value data groups. Feature point data having a UID of “0002” is stored in the second row as “feature point data B”.

An operation for initial registration in the initial registration unit 65 is performed by controlling the operation unit 37 and the display portion 31, but bidirectional communication with a separate PC or smart phone is provided via the communication unit 41, and information such as personal information or card information of a user may be input from the PC side or the smart phone side. The wearable apparatus 1 may receive the information, and store the information in the storage unit 70.

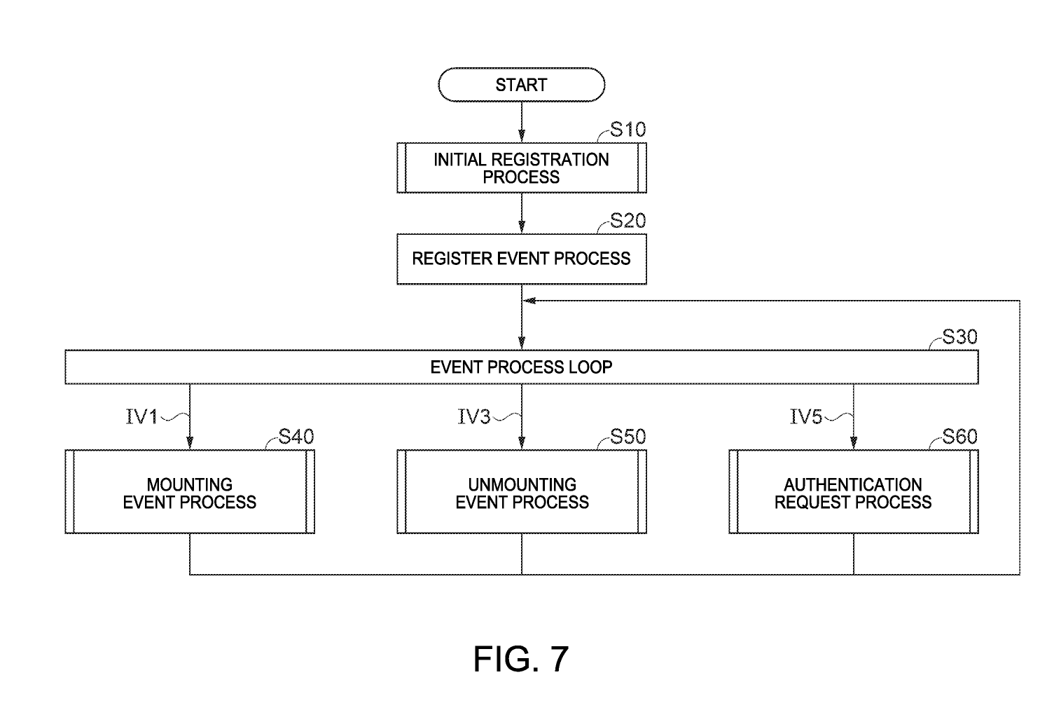

The event processing unit 67 sets a structure for controlling each functional unit according to an event driving method in the control unit 50, and activates an event process loop. Various events include, for example, an event (mounting event IV1) of detecting mounting of the wearable apparatus 1, an event (unmounting event IV3) of detecting unmounting of the wearable apparatus 1, and an event (payment request event IV5) of receiving a payment request from the charge system 5. In the event process loop, if such an event occurs, a process transitions to an event handler (not illustrated) which is set for each type of event. The event handler is a function in which a process corresponding to each event is described, and executes each functional unit as necessary so as to realize a process corresponding to an event. Flows of the process will be described later with reference to flowcharts of FIGS. 7 to 11.

The detection processing unit 52 is configured to include a mounting detection portion 53 and an unmounting detection portion 55.

The mounting detection portion 53 detects that the wearable apparatus 1 is mounted on the user's wrist, and notifies (outputs) the event processing unit 67 of the occurrence of a mounting detection event. Specifically, the mounting detection portion 53 monitors an ON/OFF signal output from the mounting unit 10. If a signal changes from OFF to ON, the mounting event IV1 is output.

If the event processing unit 67 is notified of the mounting event IV1, a process transitions to an event handler due to the notification. The event handler realizes a function of the authentication processing unit 57, and determines whether or not the user wearing the wearable apparatus 1 is a user registered in advance by the initial registration unit 65. Details of a determination method will be described in detail in the authentication processing unit 57 which will be described later. In a case where it is determined that the user is a registered user, a UID of the user is stored in result data 85 (FIG. 6).

Here, with reference to FIG. 6, the result data 85 will be described. FIG. 6 is a diagram illustrating an example of result data. A column 85A, a column 85B, a column 85C, and a column 85D are shown in the result data 85. “0001” indicating a user personally authenticated is stored in the column 85A as a UID, “1” indicating that a collation result is consistent in the column 85B as a “collation result”, collation time data is stored in the column 85C as a “collation time”, and “1” indicating that the content of the collation result is valid is stored in the column 85D as a “valid flag”.

In a case where it is determined that the user is not a registered user, the UID stored in the column 85A of the result data 85 is deleted to be “NULL” data (for example, “−1”), and the contents of the other columns 85B, 85C, and 85D are also initialized to “NULL”.

In the above-described way, in a case where the user wearing the wearable apparatus 1 is personally authenticated by the event handler of the mounting event IV1, the UID, the collation result “1”, and the valid flag “1” are stored in the result data 85, and, in a case where the user is not personally authenticated, the result data 85 is initialized.

The unmounting detection portion 55 detects that the wearable apparatus 1 is detached from the user's wrist, and notifies (outputs) the event processing unit 67 of the occurrence of the unmounting event IV3. Specifically, the unmounting detection portion 55 monitors an ON/OFF signal output from the mounting unit 10. If a signal changes from ON to OFF, the unmounting event IV3 is output.

If the event processing unit 67 is notified of the unmounting event IV3, a process transitions to an event handler due to the notification. The event handler updates the valid flag in the column 85D (FIG. 6) of the result data 85 to “0”. The valid flag “0” indicates that a user wearing the wearable apparatus 1 is not personally authenticated (invalid).

The detection processing unit 52 and the biological information measurement unit 20 correspond to a detection unit detecting a mounting state.

The request detection processing unit 56 monitors received data in the communication unit 41, and notifies (outputs) the event processing unit 67 of the payment request event IV5 in a case where the received data is information for requesting card information. The request detection processing unit 56 manages information regarding a payment process system (the charge system 5 or the like) or a card company and a service which can receive a payment process in the wearable apparatus 1 and the user. The information is stored in advance in the storage unit 70 as a request reception source list (not illustrated). The content of the request reception source list is updated on the basis of information delivered from an information source via a network.

The request detection processing unit 56 determines whether or not received data which is received from the communication unit 41 is data included in the request reception source list, and outputs the payment request event IV5 to the event processing unit 67 along with the content of the received data in a case where the received data is included in the request reception source list.

If the event processing unit 67 is notified of the payment request event IV5, a process transitions to an event handler due to the notification. The event handler executes the determination processing unit 59, and determines whether personal authentication for the user wearing the wearable apparatus 1 is valid or invalid. In a case where the personal authentication for the user is valid, a response to the received data is generated, and is transmitted to the charge system 5 via the communication unit 41. For example, in a case where the received data is information for requesting card information, the response to the received data is information including the card information. The content of received data or transmission and reception procedures depend on an arrangement with a card company or over a service, and thus the content and transmission and reception procedures corresponding to each company or service are employed.

In a case where the user wears the wearable apparatus 1, the authentication processing unit 57 performs a personal authentication process. The authentication processing unit 57 is a functional unit which is called and realized by an event handler which is executed due to a notification of the mounting event IV1. The authentication processing unit 57 controls the biological information measurement unit 20 (imaging sensor 21) so as to acquire a vein image signal as biological information. Feature point data of a vein pattern is calculated on the basis of the acquired vein image signal. This process is the same as the process performed by the initial registration unit 65. In other words, image signals having the same vein configuration are applied to the initial registration unit 65 and the authentication processing unit 57, and thus the same feature point data is calculated therein. The authentication processing unit 57 stores the calculated feature point data in the storage unit 70 as acquired authentication data 83.

The authentication processing unit 57 collates the feature point data of the registered authentication data 81 registered by the initial registration unit 65 with the feature point data of the acquired authentication data 83. For example, a determination using a correlation between both of feature point data groups is used for the collation, and a correlation coefficient is calculated. In a case where the correlation coefficient is equal to or more than a predetermined value, a strong correlation is estimated, and thus it is determined that a collation result is consistent.

In a case where the calculated correlation coefficient does not reach a predetermined value as a collation result, the authentication processing unit 57 controls the operation unit 37 and the display portion 31 so as to prompt the user to adjust a mounting position of the mounting unit 10 of the wearable apparatus 1 or to perform fine adjustment of an imaging position in the imaging sensor 21, and repeatedly performs the above-described collation process. In a case where the correlation coefficient does not reach a predetermined value as a result of repeatedly performing the collation process for a predetermined number of times or more, it is determined that a collation result is not consistent. In the authentication processing unit 57, this process depends on the user's operation, and thus time until a result of a collation result being consistent is obtained (the user is personally authenticated) is not constant but inconstant.

The authentication processing unit 57 may control the operation unit 37 and the display portion 31 so as to prompt the user to input a password or any text or figure which only the user knows. The password or any text or figure is compared and collated with a password registered in advance in the storage unit 70 of the wearable apparatus 1. This method and the collation using biological information may be combined for comparison and collation.

The authentication processing unit 57 may control the communication unit 41 so as to be connected to an authentication server provided on a computer communication network. Specific information which is specific to the user is stored in the authentication server by the user. The specific information may be any information as long as the information is set by the user. For example, the specific information may be information such as the above-described biological information, password, any text or figure, and a question and an answer set by the user in advance. The authentication processing unit 57 collates information acquired from the user wearing the wearable apparatus 1 with the specific information stored in the authentication server so as to determine consistency or inconsistency. Personal authentication in the authentication server may be performed in an overlapping manner with collation using the biological information or password.

The authentication processing unit 57 corresponds to an authentication unit.

In a case where a payment request is received from a payment process system such as the charge system 5, the determination processing unit 59 determines whether personal authentication for the user wearing the wearable apparatus 1 is valid or invalid. The determination processing unit 59 is a functional unit which is called and realized by an event handler which is executed due to a notification of the payment request event IV5. The determination processing unit 59 outputs a determination result according to the following (procedure 1) to (procedure 3) by referring to the result data 85.

The data of the result data 85 is read to an internal variable. (procedure 1)

It is determined that the column 85A (UID) is not “NULL”. (procedure 2)

It is determined whether or not the column 85B (collation result) is “1”, and the column 85D (valid flag) is “1”. (procedure 3)

In the procedure 1, a process of reading the data from the storage unit 70 to the internal variable is performed. In the procedure 2, a process of comparing the internal variable with a numerical value is performed. If the procedure 2 is established, a process in the procedure 3 is performed, that is, in the procedure 3, a process of comparing the internal variable with a numerical value is performed twice. In a case where the procedure 3 is established, it is determined that personal authentication for the user is valid, and, in a case where the procedure 2 or the procedure 3 is not established, it is determined that personal authentication for the user is invalid.

As mentioned above, process steps for realizing the procedure 1 to the procedure 3 can be realized with a constant number of steps, and a process time can be calculated on the basis of a processing speed (million instructions per second (MIPS)) of a CPU and is constant.

The determination processing unit 59 corresponds to a determination unit.

The reception processing unit 61 and the notification processing unit 63 are functional units functioning as a pair. The reception processing unit 61 controls the operation unit 37 and the notification unit 30 so as to receive an unmounting instruction. A result of personal authentication is invalid at the time of the wearable apparatus 1 being detached (unmounted), and the notification processing unit 63 outputs a warning sound such as an alarm in a case where an unmounting instruction is not received. With this configuration, in a case where the wearable apparatus 1 is detached without the user giving an unmounting instruction, a warning is issued against a detachment action contrary to the user's intention.

Specifically, the unmounting instruction is a predetermined operation performed on the wearable apparatus 1 by the user. The predetermined operation is an operation in a state in which the user mounts the wearable apparatus 1 on the wrist, and is, for example, an operation of inputting a simple password. In a case where a predetermined operation corresponding to an unmounting instruction is performed, the reception processing unit 61 determines whether or not the predetermined operation is established. For example, in a case where a simple password is input, it is determined whether or not the password matches a password set in advance in the storage unit 70. In a case where the predetermined operation is established, an unmounting flag (not illustrated) stored in the storage unit 70 is set to “ON” (indicating that unmounting is possible). Regarding the unmounting flag, in a case where the authentication processing unit 57 is called by an event handler for the mounting event IV1 when the wearable apparatus 1 is mounted, and then personal authentication is performed, the unmounting flag is set to “OFF” (indicating that unmounting is not possible).

If a notification of the unmounting event IV3 is output to the event processing unit 67 in a state in which the unmounting flag is set, an event handler executed due to the notification updates the valid flag in the column 85D (FIG. 6) of the result data 85 to “0” as described above. In other words, a result of personal authentication becomes invalid. The event handler activates the notification processing unit 63 after the process.

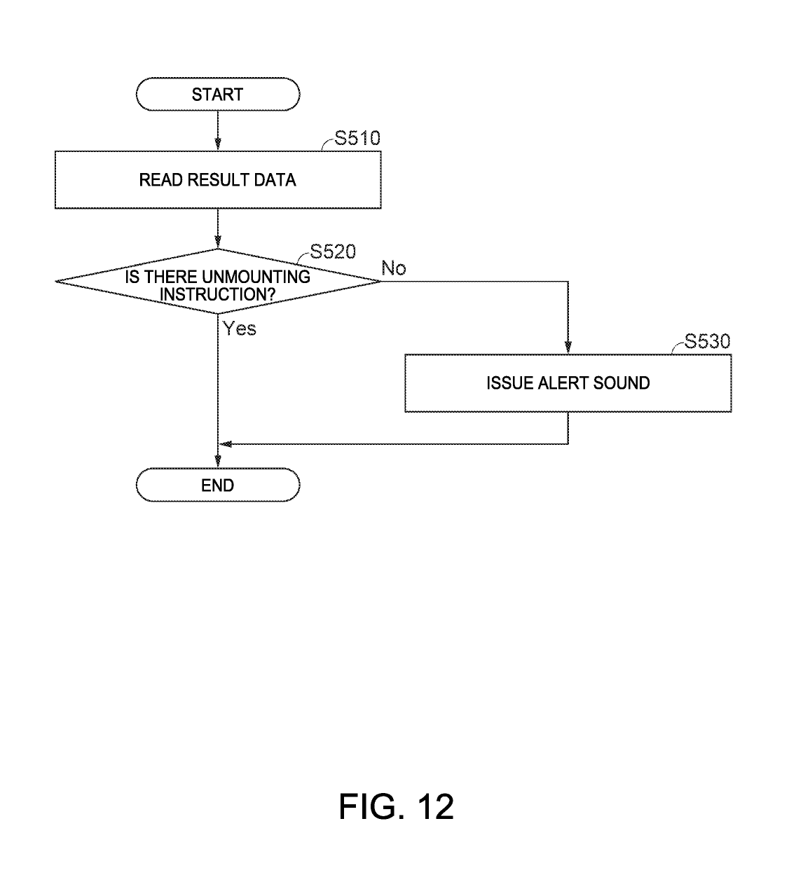

The notification processing unit 63 reads the content of the unmounting flag, and designates a volume and outputs a warning sound to the sound output portion 33 in a case where the unmounting flag is not set to “ON” (including “OFF”). The volume of a warning sound is a volume to stimulate an unspecified large number of auditory senses, and the warning sound may be an alarm sound or a sound signal such as the content that “the wearable apparatus 1 is stolen”, for example. A display signal indicating a warning may be output to the display portion 31. In this case, display to stimulate an unspecified large number of visions, such as blinking of the display portion 31, is preferably performed. A vibration signal may be output to the vibration portion 35. In this case, it is preferable that a vibration amount of the vibration portion 35 is increased, and, if a person other than the user takes the wearable apparatus 1 away, it is difficult for the person to wear the wearable apparatus by stimulating the tactile sense thereof. There may be a configuration in which a terminal (not illustrated) for outputting electric stimulation to a part of the human body such as a skin, and a tactile sense at the output part is strongly stimulated. A flow of this process will be described later with reference to FIG. 12.

A case where the unmounting flag is not set to “ON” corresponds to the time of non-reception, and a warning sound, an alarm sound, or a display signal or a vibration signal indicating a warning corresponds to abnormality information. The reception processing unit 61 corresponds to a reception unit, and the notification processing unit 63 corresponds to a notification unit.

The storage unit 70 is formed of a storage device such as a read only memory (ROM), a flash ROM, a random access memory (RAM), or the Ferroelectric RAM (FeRAM) (registered trademark), and stores various programs (not illustrated) including a control program for realizing each functional unit of the control unit 50, data, and the like. The storage unit has a work area in which data under processing in various processes, variables, values of flags, process results, and the like are temporarily stored.

The storage unit 70 stores the personal information table 72, the registered authentication data 81, the acquired authentication data 83, the result data 85, and the like.

The personal information table 72 includes personal information data 73 and card information data 75, and the personal information data 73 and the card information data 75 of users including the user stored by the initial registration unit 65 as described above are stored therein.

Feature point data of users including the user registered by the initial registration unit 65 as described above is stored in the registered authentication data 81.

Feature point data calculated by the authentication processing unit 57 as described above is stored in the acquired authentication data 83.

A UID of the user personally authenticated for the wearable apparatus 1, a collation result, a collation time, and a valid flag are stored in the result data 85 as described above.

The wearable apparatus 1 may be provided with a power generation unit (not illustrated). The power generation unit of the wearable apparatus 1 generates power for operating the wearable apparatus 1. The control unit 50 is operated by power from the power generation unit, and processes various pieces of information. The respective units such as the mounting unit 10, the biological information measurement unit 20, the notification unit 30, the operation unit 37, the clocking unit 39, the communication unit 41, and the storage unit 70 controlled by the control unit 50 are operated by power from the power generation unit. Power generation in the power generation unit may be realized by solar power generation (solar cell), and may be realized by vibration power generation, manually winding power generation, or temperature difference power generation.

Power supplied from the power generation unit to the wearable apparatus 1 is not necessarily limited to generated power. For example, if a battery can be set not to be replaced for one year or more, power supplied to the wearable apparatus 1 may be power from a button battery built into a typical watch (wristwatch) or the same type battery. The power generation unit may be a charging type secondary battery which can be charged in a noncontact manner or via a charging terminal.

### (Flow of Main Control Process)

FIG. 7 is a flowchart illustrating a flow of a main control process. Hereinafter, a description will be made as appropriate with reference to FIG. 7 along with other drawings. The following flow is a flow of the main control process, and is executed by the control unit 50 controlling the respective units including the mounting unit 10 or the biological information measurement unit 20 on the basis of a control program (not illustrated) stored in the storage unit 70.

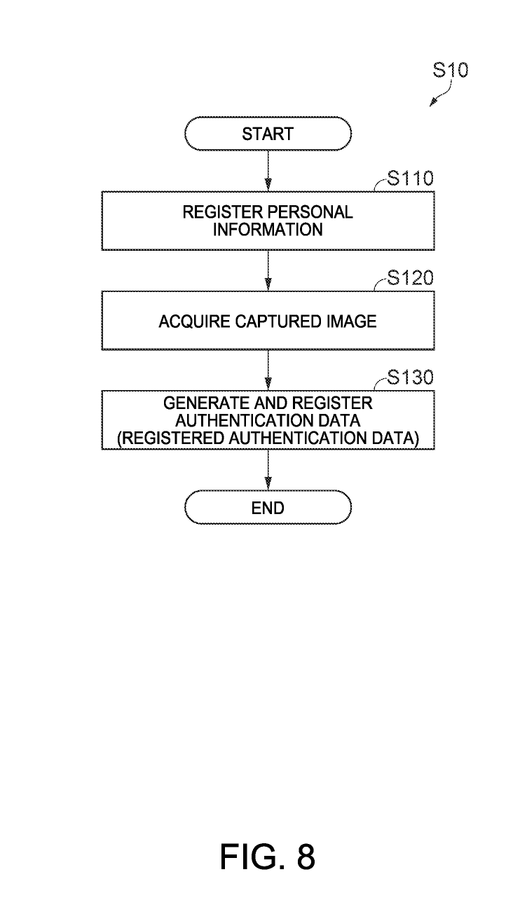

In step S10, an initial registration process is performed. Details of this process will be described in a flow illustrated in FIG. 8. In a case where the initial registration process is performed, information of the user is stored in the personal information table 72 and the registered authentication data 81.

In step S20, an event process is registered. Various events required to program an event driving method are defined, and an event handler corresponding to each event is set to be activated. Herein, events such as the mounting event IV1, the unmounting event IV3, and the payment request event IV5 used in the subsequent steps are defined, an event handler corresponding to each event is set.

Step S30 is an event process loop. Each event handler (steps S40, S50, and S60) is executed according to the occurrence of various events. The events are queued, and the event handlers are executed in an order of the occurrence of the events (a certain emergency event is prioritized). If a process in an event handler is completed, the flow returns to the main step, and waits for an event to occur.

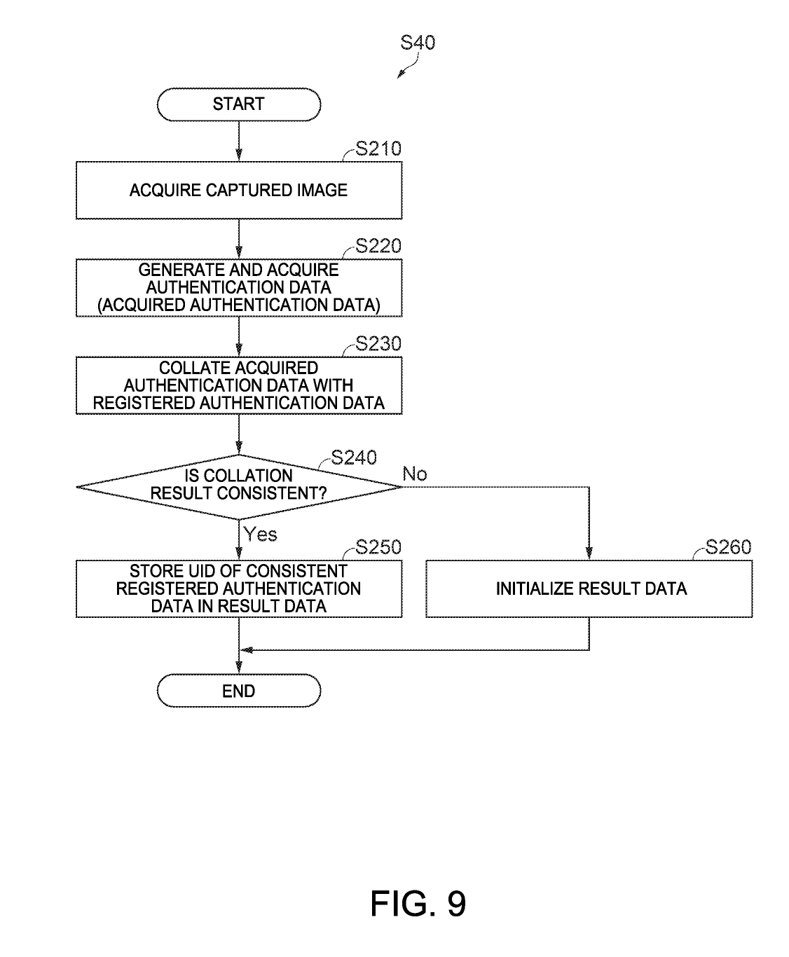

Step S40 is an event handler processed in a case where an event such as the mounting event IV1 occurs. Details of the process will be described in a flow illustrated in FIG. 9. If the process is performed, the acquired authentication data 83 is generated, and a result of collating the registered authentication data 81 with the acquired authentication data 83 is stored in the result data 85.

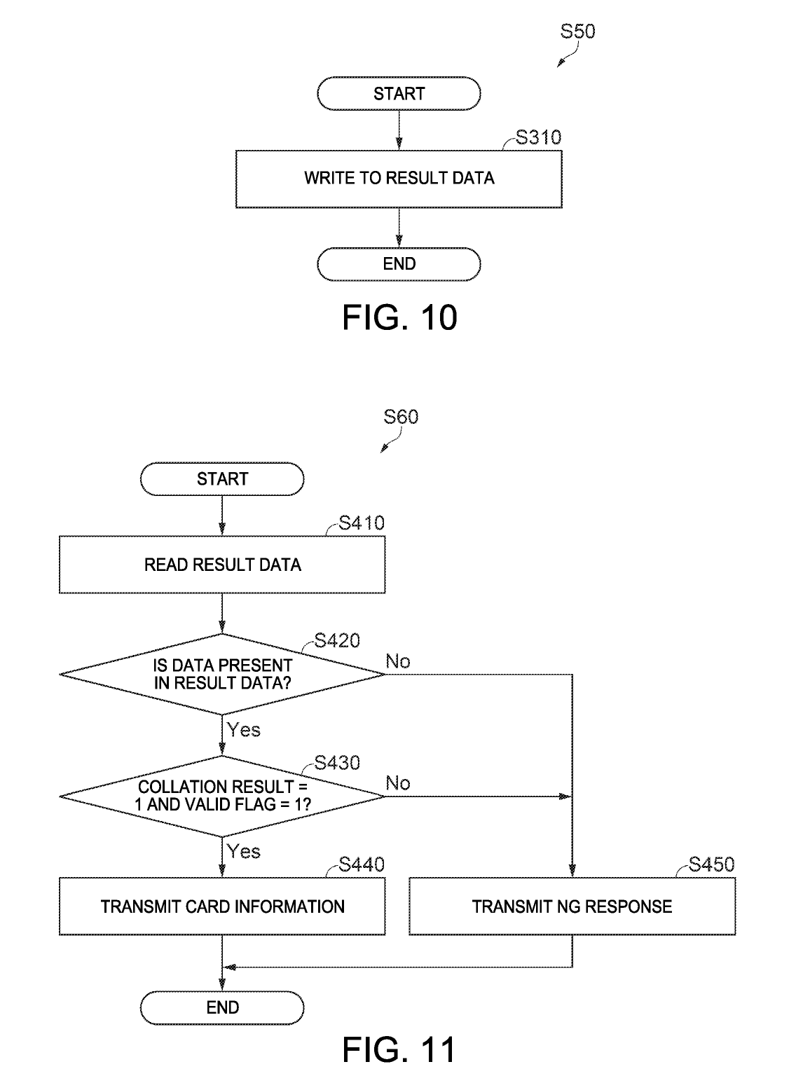

Step S50 is an event handler processed in a case where an event such as the unmounting event IV3 occurs. Details of the process will be described in a flow illustrated in FIG. 10. If the process is performed, it is recorded that a collation result in the result data 85 is invalid.

Step S60 is an event handler processed in a case where an event such as the payment request event IV5 occurs. Details of the process will be described in a flow illustrated in FIG. 11. If the process is performed, a collation result in the result data 85 is consistent, and card information or the like is transmitted in a case where the collation result is valid.

### (Flow of Initial Registration Process)

FIG. 8 is a flowchart illustrating a flow of the initial registration process. This flow corresponds to details of the process in step S10 of the main control process illustrated in FIG. 7.

In step S110, personal information is registered. The personal information data 73 and the card information data 75 of a user using the wearable apparatus 1 are acquired, and are stored in the personal information table 72.

In step S120, a captured image is acquired. The imaging sensor 21 is controlled to acquire a vein image of the user.

In step S130, authentication data is generated and is registered. Feature point data of a vein pattern is calculated on the basis of the vein image, and is registered (stored) in the registered authentication data 81. The feature point data registered in the registered authentication data 81 is feature point data for identifying an individual of the same user.

### (Flow of Mounting Event Process)

FIG. 9 is a flowchart illustrating a flow of the mounting event process. This flow corresponds to details of the process in step S40 of the main control process illustrated in FIG. 7.

In step S210, a captured image is acquired. Specifically, the imaging sensor 21 is controlled to acquire a vein image of the user.

In step S220, authentication data is generated and is stored. Feature point data of a vein pattern is calculated on the basis of the vein image, and is stored in the acquired authentication data 83.

In step S230, the acquired authentication data 83 is collated with the registered authentication data 81.

Specifically, the feature point data stored in the acquired authentication data 83 is compared and collated with the feature point data stored in the registered authentication data 81. Specifically, a correlation coefficient of both of the feature point data groups is calculated.

In step S240, it is determined whether or not a collation result is consistent. Specifically, in a case where the correlation coefficient calculated in step S230 is equal to or more than a predetermined value, it is determined that a collation result is consistent (Yes), and the flow proceeds to S250, and, in a case where the correlation coefficient is less than the predetermined value, it is determined that the collation result is not consistent (No), and the flow proceeds to S260. In a case where feature point data of a plurality of users is registered in the registered authentication data 81, the process in steps S230 and S240 are repeatedly performed by changing the feature point data in the registered authentication data 81 for each user.

In step S250, a consistent UID of the registered authentication data 81 is stored in the result data 85. Specifically, a UID having feature point data of which a collation result is consistent in step S240 is acquired from the registered authentication data 81, and the UID, the collation result “1”, the collation time, and the valid flag “1” are stored in the result data 85.

In step S260, the result data 85 is initialized. Specifically, information regarding a UID stored in the result data 85 is erased. Specifically, information regarding a UID is updated to “NULL”, a collation result is changed to “0”, a collation time is changed to “NULL”, and a valid flag is changed to “0”. Since the result data 85 is referred to by the event handler (step S60, and steps S410 to S450) for the payment request event IV5, in a case where a user wearing the wearable apparatus 1 is not registered in advance, card information is guided not to be transmitted through the initialization.

### (Flow of Unmounting Event Process)

FIG. 10 is a flowchart illustrating a flow of the unmounting event process. This flow corresponds to details of the process in step S50 of the main control process illustrated in FIG. 7.

In step S310, data is written into the result data 85. Specifically, in a case where a UID is present (not “NULL”) in the result data 85, the valid flag is changed to “0”. In other words, a result of personal authentication becomes invalid.

The content of the valid flag in the result data 85 is referred to by the event handler for the payment request event IV5, and, in a case where the valid flag is not “1”, card information is not transmitted. In other words, through this step, card information is controlled not to be transmitted in a state in which the wearable apparatus 1 is unmounted.

FIG. 12 is a flowchart illustrating a flow of the unmounting event process in the same manner as in FIG. 10.

FIG. 12 illustrates a flow of an event process in a case where an unmounting instruction is referred to after the process in FIG. 10 is performed. Specifically, the flow is executed in a case where the function of the reception processing unit 61 is realized, and the unmounting flag is set to “ON” or “OFF”.

Step S510 is the same as step S310.

In step S520, it is determined whether or not there is an unmounting instruction. Specifically, in a case where the unmounting flag is “ON” (Yes), this flow is finished, and, in a case where the unmounting flag is not “ON” (No), the flow proceeds to step S530.

In step S530, an alert sound is issued for a warning.

Through the flow, in a case where the unmounting flag is not set to “ON”, and the wearable apparatus 1 is unmounted (detached), the reception processing unit 61 issues an alert sound for prompting a warning.

### (Flow of Authentication Request Process)

FIG. 11 is a flowchart illustrating a flow of the authentication request process. This flow corresponds to details of the process in step S60 of the main control process illustrated in FIG. 7.

In step S410, the result data 85 is read. Specifically, the content of the result data 85 is read to an internal variable. The UID, the collation result, the collation time, and the valid flag are read.

In step S420, it is determined whether or not data is present in the result data 85. Specifically, in a case where the UID is not “NULL”, it is determined that data is present (Yes), the flow proceeds to step S430. In a case where the UID is “NULL”, it is determined that data is not present (No), the flow proceeds to step S450.

In step S430, it is determined whether or not the collation result is “1”, and the valid flag is “1”. In a case where the collation result is “1”, and the valid flag is “1” (Yes), the flow proceeds to step S440. In a case where one of the collation result and the valid flag is not “1” (No), the flow proceeds to step S450.

In step S440, card information is transmitted. Specifically, card information corresponding to a number of the UID read in step S410 is acquired by referring to the personal information table 72. The acquired card information is transmitted to the charge system 5 via the communication unit 41.

In step S450, a no good (NG) response is transmitted. Specifically, an NG response indicating that card information is not transmitted is transmitted to the charge system 5 via the communication unit 41.

In steps S440 and S450, card information and an NG response are transmitted, but, actually, various pieces of information are transmitted and received in a bidirectional manner on the basis of a communication type, a communication procedure, and a specification of transmission and reception data determined in advance for the wearable apparatus 1 and the charge system 5. Security measures such as encryption are also taken.

Since the respective flows illustrated in FIGS. 7 to 11 are executed, in a case where a payment process is received from the charge system 5 via the communication unit 41, a notification of the payment request event IV5 is sent, and card information is transmitted through the processes in the authentication request process (step S60, and steps S410 to S450) which is a corresponding event handler. In this case, in a case where both of a collation result and a valid flag are “1” (step S430), card information is transmitted (step S440). The collation result “1” indicates that a user is personally authenticated, and the valid flag “1” indicates that the state is maintained. The contents of the collation result and the valid flag are set through the mounting event process (step S40) and the unmounting event process (step S50) which are performed at a timing which is different from the authentication request process. Therefore, in the authentication request process, a result of an authentication process can be determined by performing the processes in steps S410, S420, and S430 after receiving a payment process. The processes in steps S410, S420, and S430 are simple memory access process and numerical value comparison process, and do not include a user's operation of which a process time is not constant, and generation (step S220) of authentication data or a comparison collation process (step S230) on feature point data requiring time for a process. In the above-described way, a payment process is received, and then a result of personal authentication for a user wearing the wearable apparatus 1 is output in restricted steps such as steps S410, S420, and S430.

As described above, the wearable apparatus 1 according to the present embodiment can achieve the following effects.

The mounting unit 10 and the detection processing unit 52 detect a mounting state of the wearable apparatus 1. Specifically, in a case where the wearable apparatus 1 is mounted, the mounting event IV1 is output, and, in a case where the wearable apparatus 1 is unmounted, the unmounting event IV3 is output.

If the mounting event IV1 is output, a corresponding event handler calls the authentication processing unit 57, and performs a personal authentication process on a user wearing the wearable apparatus 1. In a case where the user is personally authenticated, a UID of the user is stored in the result data 85. It is possible to extract card information or personal information of the user registered in advance in the wearable apparatus 1 by using the UID. In a case where the user is personally authenticated, a collation result of “1” indicating that the user is personally authenticated and a valid flag of “1” indicating that a result of the personal authentication is valid are stored in the result data 85.

If the unmounting event IV3 is output, a corresponding event handler sets the valid flag in the result data 85 to “0”. This indicates that a result of the personal authentication becomes invalid.

The request detection processing unit 56 monitors the communication unit 41. In a case where received data which is received from an external apparatus is information for requesting a payment process in the charge system 5 or the like, the payment request event IV5 is output.

If the payment request event IV5 is output, a corresponding event handler calls and executes the determination processing unit 59. The determination processing unit 59 determines whether personal authentication is valid or invalid. The determination is realized according to the procedure 1 to the procedure 3 in which information of the result data 85 is read, and a collation result is compared with the content of the valid flag. In this case, the authentication processing unit 57 is not called. In other words, the charge system 5 side can acquire a response within a constant process time through the simple procedures 1 to 3 without waiting for a process time required for a personal authentication process and an inconstant time after a payment process is requested.

Therefore, in a case where there is a request for payment from the charge system 5, the wearable apparatus 1 can determine a collation result without performing a personal authentication process, and can thus rapidly perform a response process with respect to the payment request.

In a case where the wearable apparatus 1 is detached in a state in which an unmounting instruction is not received by the reception processing unit 61 and the notification processing unit 63 of the wearable apparatus 1, a result of personal authentication can be invalidated, and a warning sound can be output. Therefore, in a case where the wearable apparatus 1 is unmounted contrary to a user's intention, it is possible to prevent illegal use of the wearable apparatus 1.

### Embodiment 2

Embodiment 2 of the present invention will be described below with reference to the drawings. In description of the present embodiment, the same constituent element as in the above Embodiment 1 is given the same reference numeral, and description thereof will be omitted or made briefly.

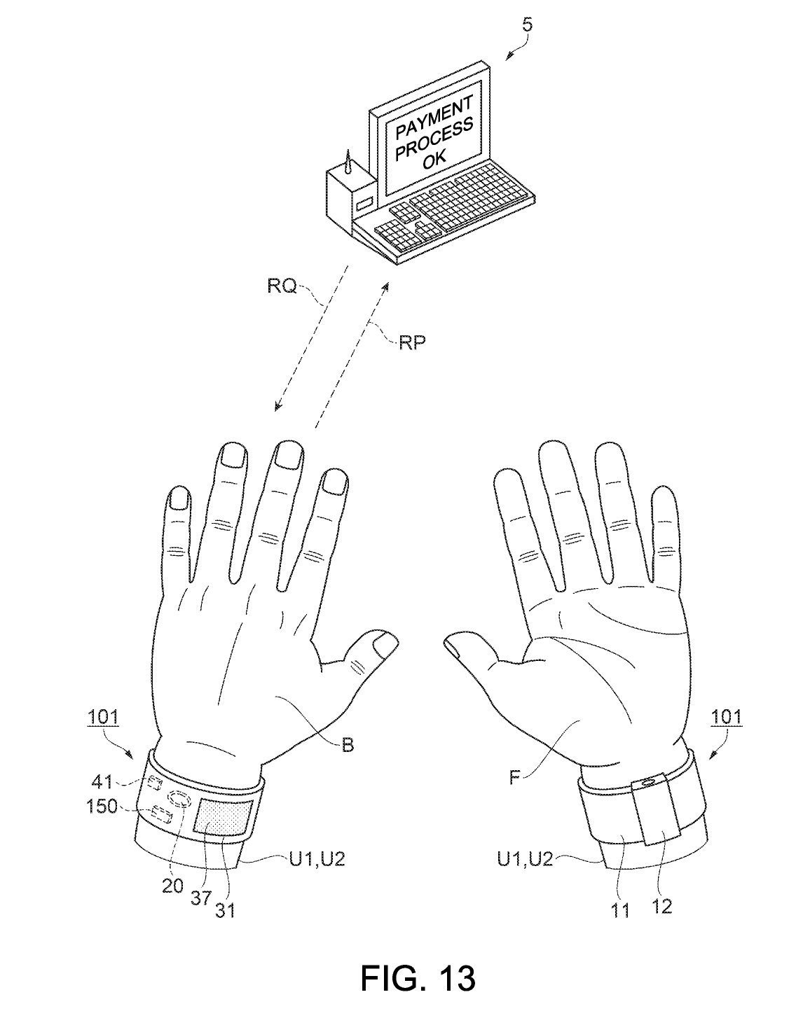

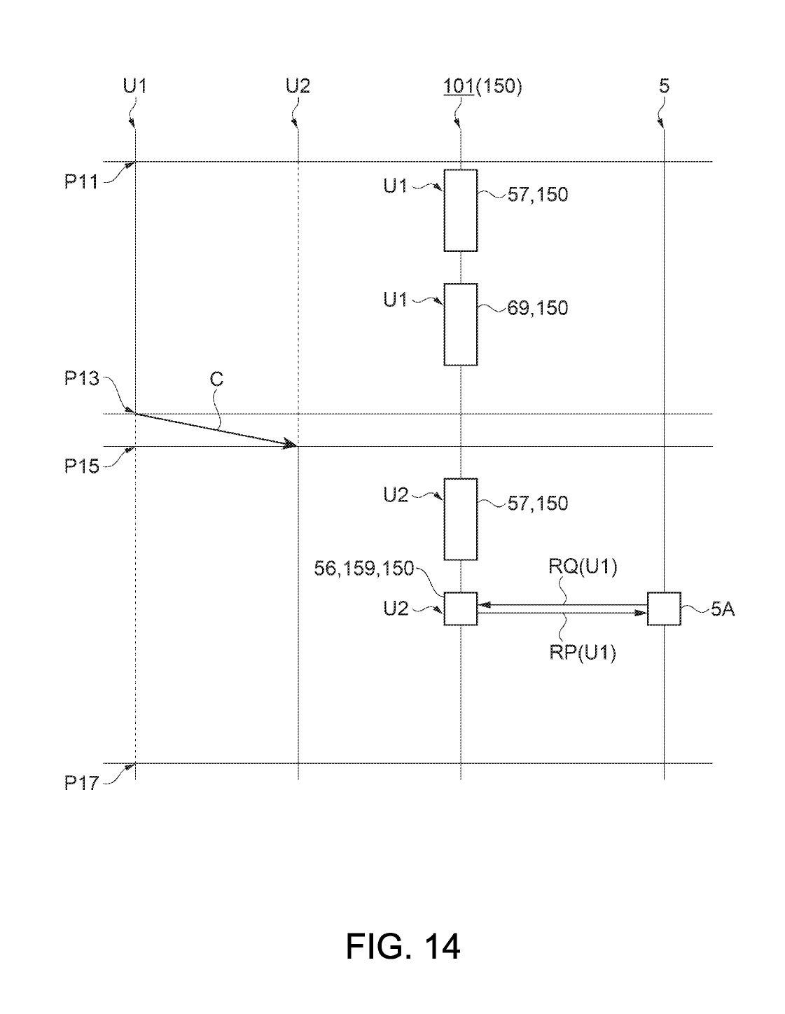

FIG. 13 is an explanatory diagram illustrating an overview of a usage form of a wearable apparatus. FIG. 14 is an explanatory diagram illustrating an overview of an authentication sequence.

A wearable apparatus 101 is a terminal mounted on the wrists of a user U1 as a first user and a user U2 as a second user (or a substitute). In the wearable apparatus 101, a display portion 31 of a flexible type of being able to be flexibly deformed which performs various displays and an operation unit 37 of a touch panel type covering the display portion 31 are incorporated into a band 11 wound on the circumference of the wrist. The wearable apparatus 101 illustrated in FIG. 13 is mounted on the left wrist of each of the users U1 and U2, includes the display portion 31 and the operation unit 37 on the back of the hand B side, and includes an opening/closing portion 12 attaching and fixing the band 11 to the wrist the flat of the hand F of the same left hand. A control unit 150 which is a central processing unit (CPU), a biological information measurement unit 20 which measures biological information of the users (hereinafter, a simply referred to as a user in a case where the user U1 and the user U2 are not differentiated from each other), a communication unit 41 which transmits and receives various pieces of data through wireless communication, and the like are built into the band 11 along with associated circuits. The user corresponds to a person using the wearable apparatus.

A charge system 5 performs a payment process (corresponding to a financial process) of the charge for a service which is used on the basis of financial information enabling a financial process to be performed, such as card information of a cash card, a credit card, a debit card, or the like, or account information in net banking or the like. The charge system 5 is installed at a location where the charge is paid, and transmits, for example, a command RQ for requesting card information to the wearable apparatus 101 through wireless communication if the user U1 or the user U2 wearing the wearable apparatus 101 comes close thereto. In a case where the command RQ is received, the wearable apparatus 101 transmits a command RP including the card information to the charge system 5.

The wearable apparatus 101 and the charge system 5 correspond to a settlement system.

In the wearable apparatus 101, card information of the user U1 is stored in a storage unit 170 (which will be described later). The wearable apparatus 101 has a function of entrusting a payment process to the user U2 which is another user with card information of the user U1 in a case where the user U1 cannot perform a payment process for the charge system 5 for a certain reason. As such a relationship between the user U1 and the user U2, for example, there is a relationship that the user U2 is a guardian of the user U1, or a relationship that the user U2 is a legal representative in all affairs regarding the property of the user U1. However, this case is only an example, and the user U2 is a third party trusted by the user U1. The charge system 5 is not limited to a financial process, and may be any system as long as the system requires identity verification. For example, there may be a system which issues a ticket or delivers an article reserved by the user U1 after identity verification, or a system which provides a document or information regarding the user U1 after identity verification.

With reference to FIG. 14, a description will be made of a inheritance process of the wearable apparatus 101 and an authentication process with the charge system 5. FIG. 14 is a diagram illustrating an overview of a inheritance and authentication sequence.

A sequence diagram of FIG. 14 illustrates mutually cooperating portions according to a flow of time (from the top to the bottom) in an operation (U1) performed by the user U1, an operation (U2) performed by the user U2, a process in the control unit 150 of the wearable apparatus 101, and a process in the charge system 5. A process method illustrated in this sequence corresponds to a settlement method. As will be described later in detail, the control unit 150 has an authentication processing unit 57, a inheritance processing unit 69, a request detection processing unit 56, and a determination processing unit 159 as functional units.

If the user U1 wears the wearable apparatus 101 (operation P11), the wearable apparatus 101 performs a personal authentication process on the user U1 (authentication processing unit 57). In the personal authentication process, a vein pattern of the user U1 registered in the wearable apparatus 101 in advance is collated with a vein pattern analyzed from a vein captured image generated by controlling an imaging sensor 21. Performing a personal authentication process on the user U1 corresponds to “a first user wearing a wearable apparatus and performing personal authentication”.

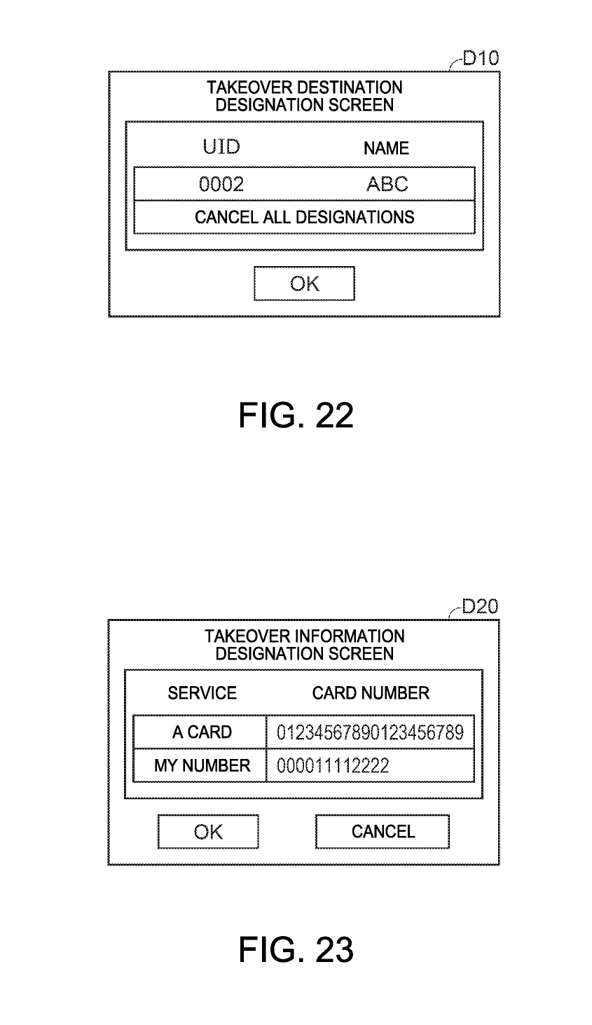

If the user U1 is personally authenticated, next, the user U1 operates the wearable apparatus 101 so as to perform a inheritance process (inheritance processing unit 69). In the inheritance process, the user U1 performs a inheritance operation to the user U2. A inheritance destination designation screen (screen D10) illustrated in FIG. 22 is an example of a screen displayed on the display portion 31 during an operation performed by the user U1. This inheritance process operation is an operation (right inheritance instruction) of taking over the right to use card information of the user U1 to the user U2 by using the wearable apparatus 101 in which the user U1 is personally authenticated. Performing a inheritance process corresponds to “a first user taking over the right of a wearable apparatus to a second user”.

The user U1 detaches and unmounts the wearable apparatus 101 from the wrist (operation P13). The user U1 delivers the wearable apparatus 101 to the user U2 (operation C).

If the user U2 wears the wearable apparatus 101 (operation P15), the wearable apparatus 101 performs a personal authentication process on the user U2 (authentication processing unit 57). In the personal authentication process, in the same manner as in the personal authentication process on the user U1, a vein pattern of the user U2 registered in the wearable apparatus 101 in advance is collated with a vein pattern analyzed from a vein captured image generated by controlling an imaging sensor 21. Performing a personal authentication process on the user U2 corresponds to “a second user wearing a wearable apparatus and performing personal authentication”.

The user U2 is personally authenticated, and then moves near the charge system 5.

If a payment process 5A occurs in the charge system 5, a command RQ for requesting card information for the payment process is transmitted from the charge system 5 to the wearable apparatus 101. In the wearable apparatus 101, the request detection processing unit 56, the determination processing unit 159, and the control unit 150 perform a process corresponding to the command RQ, and transmit (response about) a command RP including the card information of the user U1 on the basis of the inheritance information from the user U1 to the user U2. In the above-described way, a payment process can be performed by using card information of the user U1 from the wearable apparatus 101 worn by the user U2 in the time period in which the user U2 removes and thus does not wear the wearable apparatus 101 (operation P17). Performing payment using card information of the user U1 from the wearable apparatus 101 worn by the user U2 corresponds to “a second user performing settlement by using a wearable apparatus on behalf of a first user”.

In the above-described way, the control unit 150 can entrust a payment process using card information of the user U1 which is possible as a result of the user U1 being personally authenticated to the user U2.

Hereinafter, a detailed description will be made of a configuration of the wearable apparatus 101 which can achieve the effect.

### (Configuration of Wearable Apparatus)

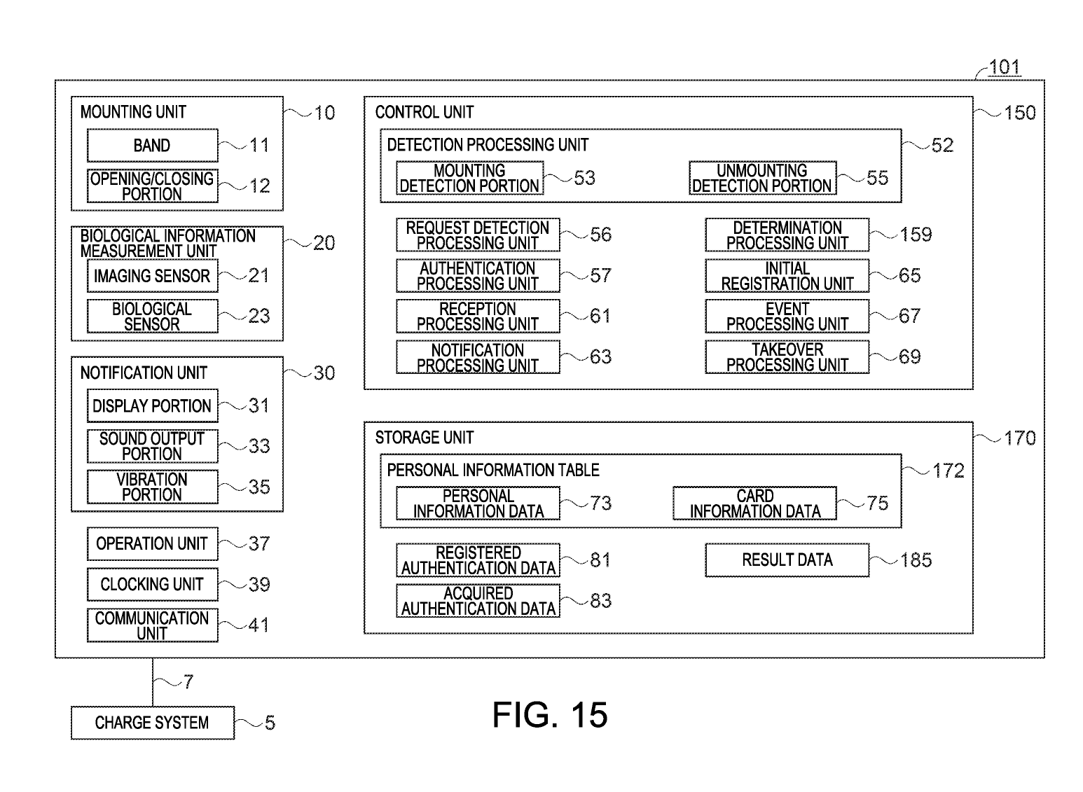

FIG. 15 is a block diagram illustrating a schematic configuration of the wearable apparatus.

The wearable apparatus 101 is configured to include a mounting unit 10, the biological information measurement unit 20, a notification unit 30, the operation unit 37, a clocking unit 39, the communication unit 41, the control unit 150, and the storage unit 170. The wearable apparatus 101 is connected to the charge system 5 via communication 7.

The wearable apparatus 101 of the present embodiment is different from the wearable apparatus 1 of Embodiment 1 in that the control unit 150 is provided instead of the control unit 50, and the storage unit 170 is provided instead of the storage unit 70. Other configurations are the same as those of the wearable apparatus 1. Therefore, hereinafter, differences from the wearable apparatus 1 will be focused.

### (Control Unit)

The control unit 150 is a control device and a calculation device which are configured to include a processor such as a CPU or a digital signal processor (DSP), and integrally control the respective units of the wearable apparatus 101. The control unit 150 realizes various functions of the wearable apparatus 101 according to various programs including a control program (not illustrated) stored in the storage unit 170.

The control unit 150 has a detection processing unit 52, a request detection processing unit 56, the authentication processing unit 57, a determination processing unit 159, a reception processing unit 61, a notification processing unit 63, and the inheritance processing unit 69 as principal functional units, and integrally controls the functional units with an event processing unit 67 and an event handler (which will be described later) so as to realize the functions of the wearable apparatus 101. The control unit 150 realizes a function of an initial registration unit 65 before the functional units are realized.

### (Initial Registration Unit)

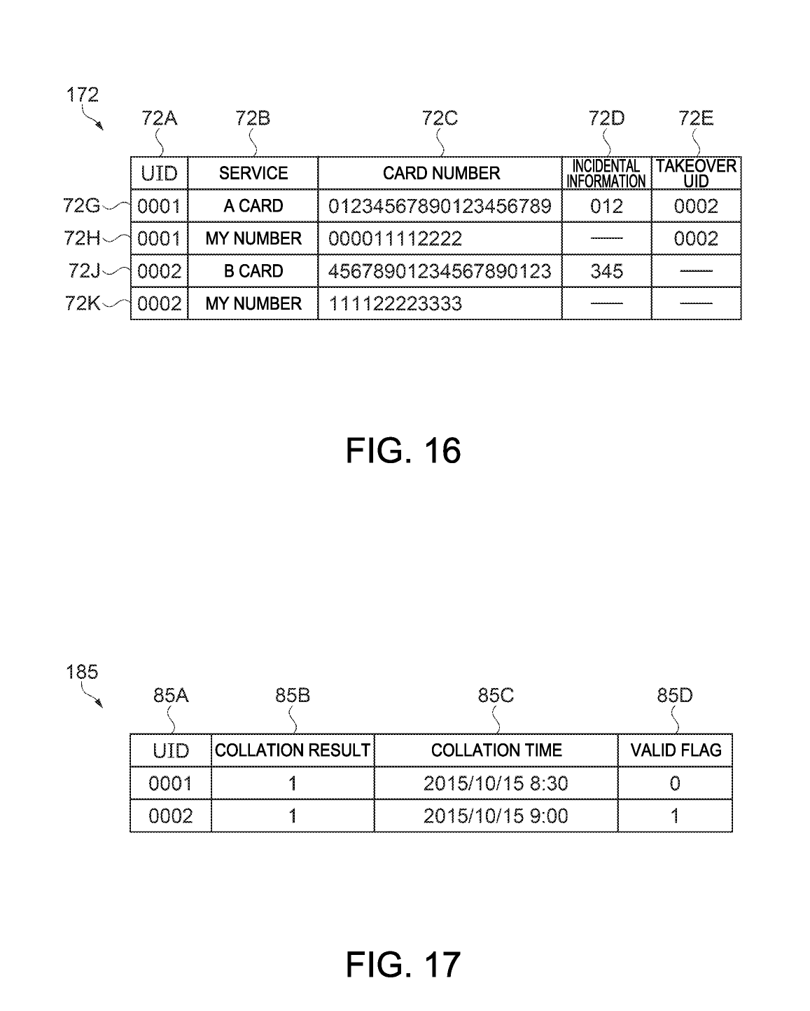

The initial registration unit 65 performs initial registration of a user using the wearable apparatus 101. Specifically, the initial registration unit 65 controls the operation unit 37 and the display portion 31 so as to acquire information such as personal information or card information of the user. The acquired information is stored in the storage unit 170 as a personal information table 172 (FIG. 16). When the personal information table is stored, a user identification (UID) for identifying the user in the control unit 150 is generated and is managed as information regarding the user.

Here, with reference to FIG. 16, the personal information table 172 will be described. FIG. 16 is a diagram illustrating an example of a personal information table. A column 72A, a column 72B, a column 72C, a column 72D, a column 72E, a row 72G, a row 72H, a row 72J, and a row 72K are shown in the personal information table 172. A UID is stored in the column 72A. The type of service is stored in the column 72B, a card number is stored in the column 72C, and incidental information is stored in the column 72D. A inheritance UID is stored in the column 72E. The inheritance UID is a UID stored by the inheritance processing unit 69 (details thereof will be described later). The row 72G shows that a credit card service of the user U1 having a UID of “0001” is “A card”, a card number thereof is “01234567890123456789”, and incidental information is “012”. The row 72H shows that the type of personal information of the user U1 having the UID “0001” which is the same as in the row 72G is “my number”, and a number thereof is “000011112222”. Information regarding the user U2 having a UID of “0002” is stored in the row 72J and the row 72K in the same form as the information regarding the user U1. The information regarding the user U1 stored in the personal information table 172 corresponds to first user information.

The personal information is information such as my number, a name, an age, and an address, and may be transmitted in a case of being requested from the charge system 5 or other systems. The card information is, for example, a credit card number (credit card information), a debit card information (debit card information), a cash card number (cash card information), and an account number (net banking information) used for net banking, and a security code or a password is stored as incidental information as necessary. Shared information between the user U1 and the user U2 may be stored in the personal information table 172.

The initial registration unit 65 controls the operation unit 37 and the display portion 31 so as to prompt a user to wear the wearable apparatus 101, and measures biological information for specifying the user individual after the user wears the wearable apparatus 101. The biological information is a vein image signal with high individual identification accuracy as a preferred example. The initial registration unit 65 controls the biological information measurement unit 20 so as to acquire a vein image signal. Well-known processes such as a binarization process, a thinning process, and feature point extraction of an image are applied to the acquired vein image signal, and thus feature point data of a vein pattern is calculated. The feature point data includes a plurality of pieces of feature point data such as a gap between or the number of branch points of the vein pattern, a length and a shape between branch points, and an angle of an included angle at a branch point. If the feature point data of the user is calculated, the initial registration unit 65 stores the feature point data in the storage unit 170 as registered authentication data 81 (FIG. 5) in correlation with a UID of the user.

Here, with reference to FIG. 5, the registered authentication data 81 of the present embodiment will be described. A column 81A and a column 81B are shown in the registered authentication data 81. A UID is stored in the column 81A. Feature point data of a vein pattern is stored in the column 81B. Feature point data having a UID of “0001” (user U1) is stored in the first row of the registered authentication data 81 as “feature point data A” of the present embodiment. The feature point data A is a numerical value data group indicating feature points. There may be a plurality of numerical value data groups. Feature point data having a UID of “0002” (user U2) is stored in the second row as “feature point data B”.

An operation for initial registration in the initial registration unit 65 is performed by controlling the operation unit 37 and the display portion 31, but bidirectional communication with a separate PC or smart phone is provided via the communication unit 41, and information such as personal information or card information of a user may be input from the PC side or the smart phone side. The wearable apparatus 101 may receive the information, and store the information in the storage unit 170.

### (Event Processing Unit)

The event processing unit 67 sets a structure for controlling each functional unit according to an event driving method in the control unit 150, and activates an event process loop. Various events includes, for example, an event (mounting event IV1) of detecting mounting of the wearable apparatus 101, an event (unmounting event IV3) of detecting unmounting of the wearable apparatus 101, an event (payment request event IV5) of receiving a payment request from the charge system 5, and an event (inheritance event IV7) in a case where a inheritance setting of the wearable apparatus 101 is selected through the user's operation. In the event process loop, if such an event occurs, a process transitions to an event handler (not illustrated) which is set for each type of event. The event handler which is a functional unit forming the control unit 150 is a function in which a process corresponding to each event is described, and executes each functional unit as necessary so as to realize a process corresponding to an event. Flows of a process in the event handler will be described later with reference to flowcharts of FIGS. 18 to 20 and FIG. 12.

### (Mounting Detection Portion)

The detection processing unit 52 is configured to include a mounting detection portion 53 and an unmounting detection portion 55.

The mounting detection portion 53 detects that the wearable apparatus 101 is mounted on the user's wrist, and notifies (outputs) the event processing unit 67 of the occurrence of a mounting detection event. Specifically, the mounting detection portion 53 monitors an ON/OFF signal output from the mounting unit 10. If a signal changes from OFF to ON, the mounting event IV1 is output.

### (Event Handler for Mounting Event IV1)

If the event processing unit 67 is notified of the mounting event IV1, a process transitions to an event handler due to the notification. The event handler realizes a function of the authentication processing unit 57, and determines whether or not the user wearing the wearable apparatus 101 is a user registered in advance by the initial registration unit 65. Details of a determination method will be described in detail in the authentication processing unit 57 which will be described later. In a case where it is determined that the user is a registered user, a UID of the user is stored in result data 185 (FIG. 17). Next, in a case where the user wearing the wearable apparatus takes over the right from another user (for example, the user U2 takes over the right from the user U1) (during inheritance), the event handler for the mounting event IV1 outputs information regarding the user (user U1) who is a right inheritance source to the display portion 31. In a case where the right is not taken over from any user (during non-inheritance), the event handler outputs information (non-inheritance information) indicating that the right is not taken over to the display portion 31. The display portion 31 displays each piece of information. At this time, buzzer sound data for calling attention by hearing may be output to the sound output portion 33, or vibration data for stimulating the tactile sense may be output to the vibration portion 35. The function of outputting information in the event handler, and the notification unit 30 including the display portion 31, the sound output portion 33, and the vibration portion 35 corresponds to a notification unit.

Here, with reference to FIG. 17, the result data 185 will be described. FIG. 17 is a diagram illustrating an example of result data. A column 85A, a column 85B, a column 85C, and a column 85D are shown in the result data 185. A UID is stored in the column 85A, “1” indicating that a collation result is consistent in the column 85B (a user having the corresponding UID is personally authenticated), and collation time data is stored in the column 85C. In the column 85D, “1” is stored as a valid flag in a case where the content of the collation result (the content of the column 85B) is valid, and “0” is stored as a valid flag in a case where the content thereof is not valid. In a case where it is determined that the user wearing the wearable apparatus 101 is not a user registered in advance, the UID stored in the column 85A of the result data 185 is deleted to be “NULL” data (for example, “−1”), and the contents of the other columns 85B, 85C, and 85D are also initialized to “NULL”.

In the example of the result data 185 illustrated in FIG. 17, since the valid flag for the user U1 is “0”, and the valid flag for the user U2 is “1”, this indicates that the wearable apparatus 101 is worn by the user U2.

### (Unmounting Detection Portion)

The unmounting detection portion 55 detects that the wearable apparatus 101 is detached from the user's wrist, and notifies (outputs) the event processing unit 67 of the occurrence of the unmounting event IV3. Specifically, the unmounting detection portion 55 monitors an ON/OFF signal output from the mounting unit 10. If a signal changes from ON to OFF, the unmounting event IV3 is output.

### (Event Handler for Unmounting Event IV3)

If the event processing unit 67 is notified of the unmounting event IV3, a process transitions to an event handler due to the notification. The event handler updates the valid flag in the column 85D (FIG. 17) of the result data 185 to “0”. The valid flag set to “0” indicates that a result of personal authentication for the user wearing the wearable apparatus 101 is invalid.

The mounting unit 10, the detection processing unit 52, and the biological information measurement unit 20 correspond to a detection unit detecting a mounting state.

### (Request Detection Processing Unit)

The request detection processing unit 56 monitors received data in the communication unit 41, and notifies (outputs) the event processing unit 67 of the payment request event IV5 in a case where the received data is information for requesting card information. The request detection processing unit 56 manages information regarding a payment process system (the charge system 5 or the like) or a card company and a service which can receive a payment process in the wearable apparatus 101 and the user. The information is stored in advance in the storage unit 170 as a request reception source list (not illustrated). The content of the request reception source list is updated on the basis of information delivered from an information source via a network.

The request detection processing unit 56 determines whether or not received data which is received from the communication unit 41 is data included in the request reception source list, and outputs the payment request event IV5 to the event processing unit 67 along with the content of the received data in a case where the received data is included in the request reception source list.

### (Event Handler for Payment Request Event IV5)

If the event processing unit 67 is notified of the payment request event IV5, a process transitions to an event handler due to the notification. The event handler executes the determination processing unit 159, and determines whether a result of personal authentication for the user wearing the wearable apparatus 101 is valid or invalid.

In a case where it is determined that the result is invalid, a no good (NG) response indicating that card information is not transmitted is transmitted as response information to the received data.

In a case where it is determined that the result is valid, response information to the received data is generated, and is transmitted to the charge system 5 via the communication unit 41. For example, if a specific service is designated in the received data, the response information is information regarding a card number of a row matching the content of a service stored in the column 72B of the personal information table 172. In this case, the inheritance UID in the column 72E is referred to. For example, in a case where a personally authenticated user is the user U2 (UID “0002”), and a result is determined as being valid, card information in the row 72G in which “0002” is present in the column 72E of the personal information table 172 is selected. A user having the UID stored in the column 72E has taken over the right to use the information stored in the same row. In the example of the personal information table 172, card information including the service “A card” and the card number “01234567890123456789” in the row 72G is selected. In the above-described way, the wearable apparatus 101 worn by the user U2 uses the card information of the user U1 stored in the storage unit 170 (outputs the card information to the charge system 5) in a case where the right is taken over from the user U1. The function realized by the event handler corresponds to a function of outputting information based on a result of personal authentication in the authentication unit.

Information output to the external charge system 5 is information based on a result of personal authentication, and is not limited to original data (data for specifying an individual) regarding the result of personal authentication.

The content of received data or transmission and reception procedures depend on an arrangement with a card company or over a service, and thus the content and transmission and reception procedures corresponding to each company or service are employed.

### (Authentication Processing Unit)

In a case where the user wears the wearable apparatus 101, the authentication processing unit 57 performs a personal authentication process in the same manner as in Embodiment 1. The authentication processing unit 57 corresponds to an authentication unit.

### (Determination Processing Unit)

In a case where a payment request is received from a payment process system such as the charge system 5, the determination processing unit 159 determines whether a result of personal authentication for the user wearing the wearable apparatus 101 is valid or invalid. The determination processing unit 159 is a functional unit which is called and realized by an event handler which is executed due to a notification of the payment request event IV5. The determination processing unit 159 outputs a determination result according to the following (procedure 1) to (procedure 3) by referring to the result data 185.

The data of the result data 185 is read to an internal variable row by row. (procedure 1)

It is determined that the column 85A (UID) is not “NULL”. (procedure 2)

It is determined whether or not the column 85B (collation result) is “1”, and the column 85D (valid flag) is “1”. (procedure 3)

In the procedure 1, a process of reading the data from the storage unit 170 to the internal variable is performed. In the procedure 2, a process of comparing the internal variable with “NULL” is performed. If the procedure 2 is established, a process in the procedure 3 is performed, that is, in the procedure 3, a process of comparing the internal variable with a numerical value of “1” is performed. In a case where the procedure 3 is established, a user having a corresponding UID is personally authenticated, and a result thereof is determined as being valid. In a case where the procedure 2 or the procedure 3 is not established, it is determined that a result of personal authentication for the user is invalid.

As mentioned above, process steps for realizing the procedure 1 to the procedure 3 can be realized with a constant number of steps, and a process time can be calculated on the basis of a processing speed (million instructions per second (MIPS)) of a CPU and is constant.

In the above-described determination processing unit 159, a description has been made of an example in which the determination processing unit 159 performs a final determination of whether personal authentication is valid or invalid, but this is only an example, and the determination processing unit 159 may perform a process of outputting information required to determine whether personal authentication is valid or invalid. In this case, the charge system 5 which is an external terminal performs a final determination of whether personal authentication is valid or invalid on the basis of the process in the determination processing unit 159. In both a case where a final determination of personal authentication is performed in the wearable apparatus 101 and a case where the final determination is performed in the charge system 5 which is an external apparatus of the wearable apparatus 101, whether personal authentication is valid or invalid is determined on the basis of the determination processing unit 159.

Therefore, through information processing in the determination processing unit 159, in a case where there is a payment request from the charge system 5, if a user wears the wearable apparatus 101, the wearable apparatus 101 can determine a collation result without performing a personal authentication process every time, and can thus rapidly perform a response process with respect to the payment request.

The determination processing unit 159 corresponds to a determination processing unit.

### (Reception Processing Unit and Notification Processing Unit)

The reception processing unit 61 and the notification processing unit 63 are functional units functioning as a pair in the same manner as in Embodiment 1.

In the present embodiment, if a notification of the unmounting event IV3 is output to the event processing unit 67 in a state in which the unmounting flag is set, an event handler executed due to the notification updates the valid flag in the column 85D (FIG. 17) of the result data 185 to “0”. In other words, a result of personal authentication becomes invalid. This process flow is the same as the flow illustrated in FIG. 12.

### (Inheritance Processing Unit)

The inheritance processing unit 69 performs a procedure of taking over the right to use the wearable apparatus 101 holding information regarding a personally authenticated user (user U1) to another user (user U2).

The control unit 150 controls the operation unit 37 and the display portion 31, and notifies the event processing unit 67 of the inheritance event IV7 if a predetermined operation is performed.

### (Event Handler for Inheritance Event IV7/Inheritance Processing Unit)

If the event processing unit 67 is notified of the inheritance event IV7, a process transitions to an event handler due to the notification. If the event handler is executed, a function of the inheritance processing unit 69 is realized.

The inheritance processing unit 69 corresponds to a inheritance reception unit, and receives a right inheritance instruction for the wearable apparatus 101 from a user (user U1). Specifically, the inheritance processing unit 69 outputs a inheritance destination designation screen for the user U1 selecting a user (user U2) who is a inheritance destination, and a inheritance information designation screen for designating inheritance information, to the display portion 31. The inheritance processing unit 69 controls the operation unit 37, so as to acquire an operation signal selected by the user U1.

FIG. 22 is a diagram illustrating an example of a inheritance destination designation screen. FIG. 23 is a diagram illustrating an example of a inheritance information designation screen. The inheritance destination designation screen (screen D10) and the inheritance information designation screen (screen D20) are screens displayed on the display portion 31, and the screen D20 is displayed after the screen D10 is operated. A displayed selection menu may be selected by using the operation unit 37.

The inheritance processing unit 69 refers to the registered authentication data 81 and the personal information table 172, and acquires a UID other than the user wearing the wearable apparatus 101 and related information such as the name related to the UID. The screen D10 is generated by using the acquired UID and name. On the screen D10, the UID “0002” and the name “ABC” indicating the user U2 are displayed in the first row of the selection menu, and “cancel all designations” is displayed in the second row. An “OK” button indicating determination is displayed. If the UID “0002” and the name “ABC” are selected by the user U1, the same row is inversely displayed (not illustrated), and “OK” is selected, so that the user U2 is determined as a inheritance destination. If “cancel all designations” is selected, designation of a inheritance destination is canceled.

The inheritance processing unit 69 stores the UID “0002” in an internal variable.

The inheritance processing unit 69 refers to the personal information table 172, and reads information regarding the user (the user U1 and the UID “0001”) wearing the wearable apparatus 101 so as to generate the screen D20. On the screen D20, the service “A card” and the card number “01234567890123456789” are displayed in the first row of the selection menu, and the service “my number” and the card number “000011112222” are displayed in the second row. An “OK” button indicating determination and a “cancel” button indicating cancellation are displayed. If a service which is taken over is selected through an operation of the user U1, a row of the selected service is inversely displayed (not illustrated), and, if “OK” is selected, the service taken over to the user U2 is determined. The inheritance processing unit 69 stores a UID of the inheritance destination in a row corresponding to the service determined in the column 72E (inheritance UID) of the personal information table 172. The column 72E (inheritance UID) of a row corresponding to a service associated with a menu which is not selected in the selection menu is updated to “NULL”. The example of the personal information table 172 shows a state in which both of the first row and the second row of the selection menu on the screen D20 are selected, and thus the UID “0002” is stored in the row 72G and the row 72H of the column 72E.

In a case where “cancel all designations” is selected and determined on the screen D10, the inheritance processing unit 69 updates all UIDs stored in the column 72E of the personal information table 172 to “NULL”.

### (Storage Unit)

The storage unit 170 is formed of a storage device such as a read only memory (ROM), a flash ROM, a random access memory (RAM), or the Ferroelectric RAM (FeRAM) (registered trademark), and stores various programs (not illustrated) including a control program for realizing each functional unit of the control unit 150, data, and the like. The storage unit has a work area in which data under processing in various processes, variables, values of flags, process results, and the like are temporarily stored.

The storage unit 170 stores the personal information table 172, the registered authentication data 81, the acquired authentication data 83, the result data 185, and the like.

The personal information table 172 includes personal information data 73 and card information data 75, and the personal information data 73 and the card information data 75 of users including the user stored by the initial registration unit 65 as described above are stored therein.

Feature point data of users including the user registered by the initial registration unit 65 as described above is stored in the registered authentication data 81.

Feature point data calculated by the authentication processing unit 57 as described above is stored in the acquired authentication data 83.

A UID of the user personally authenticated for the wearable apparatus 101, a collation result, a collation time, and a valid flag are stored in the result data 185 as described above.

### (Power Generation Unit)

The wearable apparatus 101 may be provided with a power generation unit (not illustrated) in the same manner as in Embodiment 1.

### (Flow of Main Control Process)

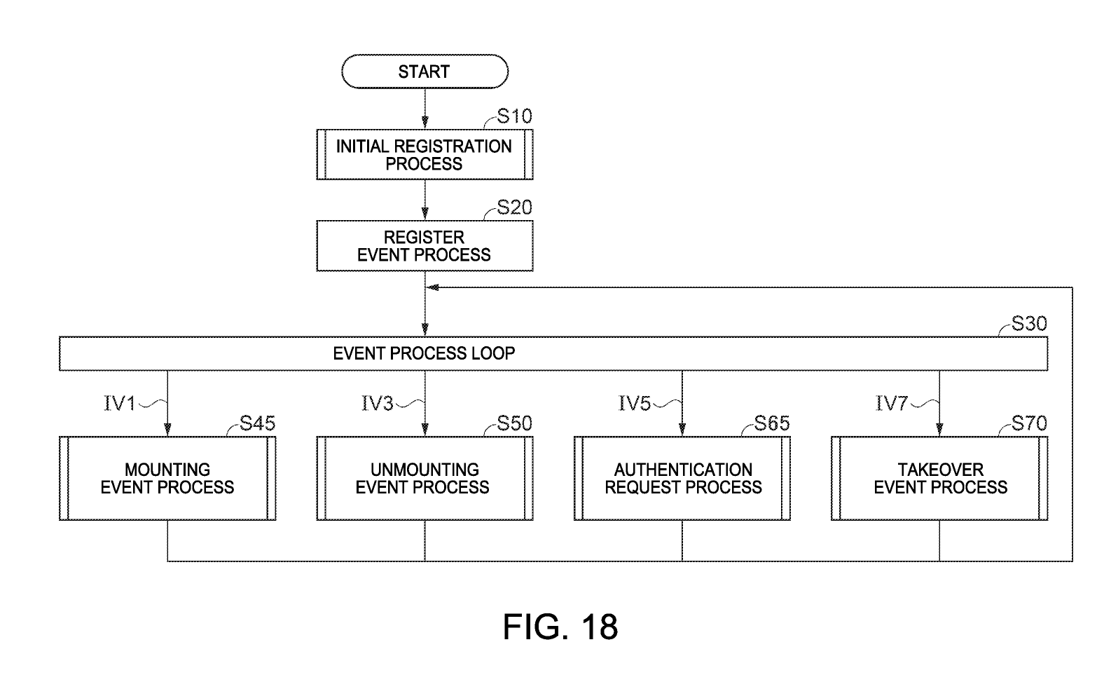

FIG. 18 is a flowchart illustrating a flow of a main control process. Hereinafter, a description will be made as appropriate with reference to FIG. 18 along with other drawings. The following flow is a flow of the main control process, and is executed by the control unit 150 controlling the respective units including the mounting unit 10 or the biological information measurement unit 20 on the basis of a control program (not illustrated) stored in the storage unit 170.

In step S10, an initial registration process is performed. Details of this process will be described in a flow illustrated in FIG. 8 in the same manner as in Embodiment 1. In a case where the initial registration process is performed, information of the user is stored in the personal information table 172 and the registered authentication data 81.

In step S20, an event process is registered. Various events required to program an event driving method are defined, and an event handler corresponding to each event is set to be activated. Herein, events such as the mounting event IV1, the unmounting event IV3, the payment request event IV5, and the inheritance event IV7 used in the subsequent steps are defined, an event handler corresponding to each event is set.

Step S30 is an event process loop. Each event handler (steps S45, S50, S65, and S70) is executed according to the occurrence of various events. The events are queued, and the event handlers are executed in an order of the occurrence of the events (a certain emergency event is prioritized). If a process in an event handler is completed, the flow returns to the main step, and waits for an event to occur.

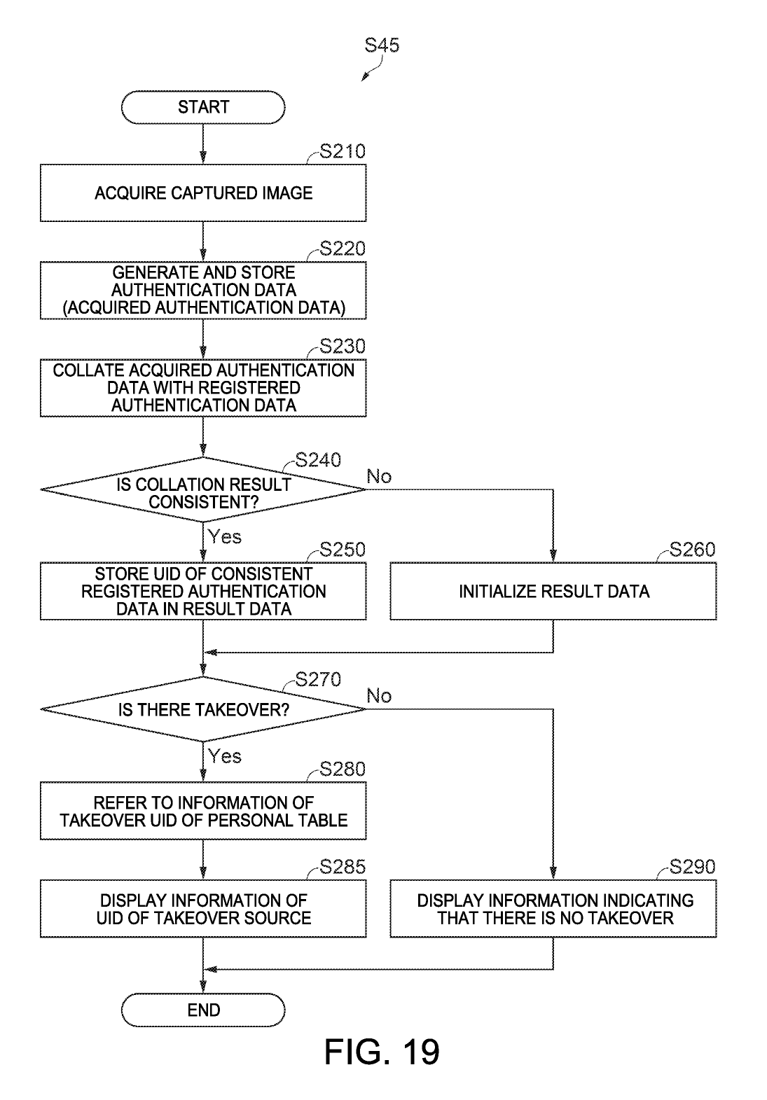

Step S45 is an event handler processed in a case where an event such as the mounting event IV1 occurs, and a mounting event process is performed. Details of the process will be described in a flow illustrated in FIG. 19. If the process is performed, the acquired authentication data 83 is generated, and a result of collating the registered authentication data 81 with the acquired authentication data 83 is stored in the result data 185.

Step S50 is an event handler processed in a case where an event such as the unmounting event IV3 occurs, and an unmounting event process is performed. Details of the process will be described in a flow illustrated in FIG. 10 in the same manner as in Embodiment 1. If the process is performed, it is recorded that a collation result in the result data 185 is invalid.

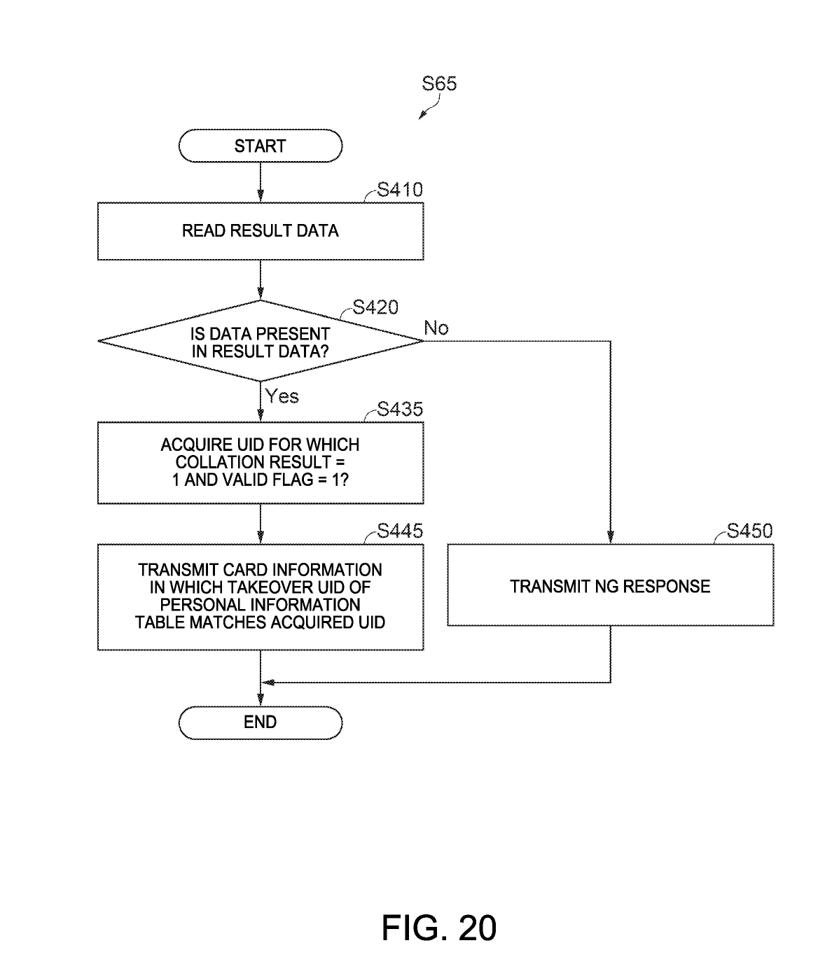

Step S65 is an event handler processed in a case where an event such as the payment request event IV5 occurs, and an authentication request process is performed. Details of the process will be described in a flow illustrated in FIG. 20. If the process is performed, a collation result in the result data 185 is consistent, and card information or the like is transmitted in a case where the collation result is valid.

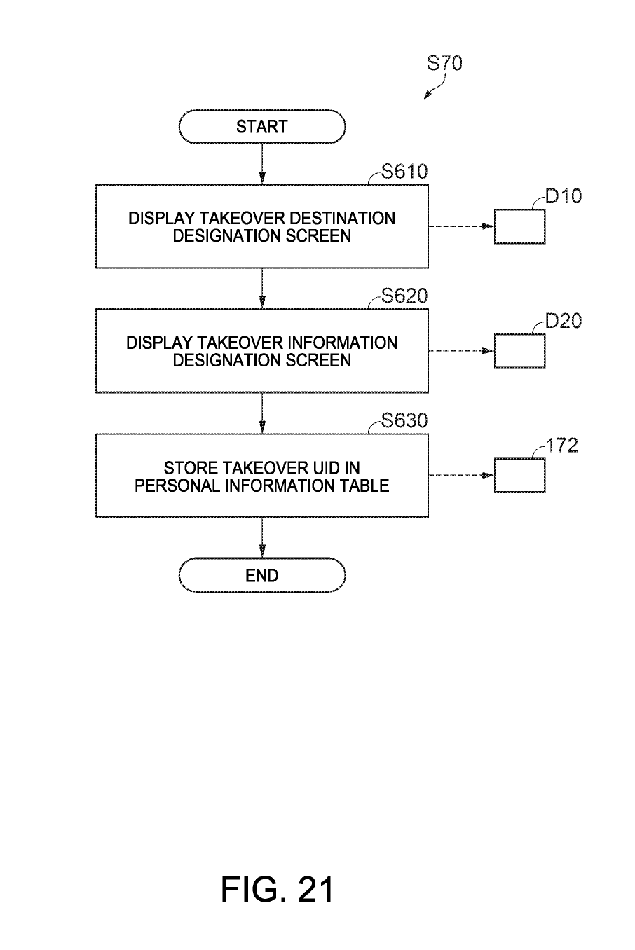

Step S70 is an event handler processed in a case where an event such as the inheritance event IV7 occurs, and a inheritance event process is performed. Details of the process will be described in a flow illustrated in FIG. 21. If the process is performed, a inheritance destination designation screen (screen D10) and a inheritance information designation screen (screen D20) are displayed on the display portion 31, and a inheritance UID is stored in the column 72E of the personal information table 172.

### (Flow of Initial Registration Process)

FIG. 8 is a flowchart illustrating a flow of the initial registration process. This flow corresponds to details of the process in step S10 of the main control process illustrated in FIG. 18.

In step S110, personal information is registered. In the present embodiment, the personal information data 73 and the card information data 75 of a user using the wearable apparatus 101 are acquired, and are stored in the personal information table 172.

In step S120, a captured image is acquired. The imaging sensor 21 is controlled to acquire a vein image of the user.

In step S130, authentication data is generated and is registered. Feature point data of a vein pattern is calculated on the basis of the vein image, and is registered (stored) in the registered authentication data 81. The feature point data registered in the registered authentication data 81 is feature point data for identifying an individual of the same user.

### (Flow of Mounting Event Process)

FIG. 19 is a flowchart illustrating a flow of the mounting event process. This flow corresponds to details of the process in step S45 of the main control process illustrated in FIG. 18.

In step S210, a captured image is acquired. Specifically, the imaging sensor 21 is controlled to acquire a vein image of the user.

In step S220, authentication data is generated and is registered. Feature point data of a vein pattern is calculated on the basis of the vein image, and is stored in the acquired authentication data 83.

In step S230, the acquired authentication data 83 is collated with the registered authentication data 81. Specifically, the feature point data stored in the acquired authentication data 83 is compared and collated with the feature point data stored in the registered authentication data 81. Specifically, a correlation coefficient of both of the feature point data groups is calculated.

In step S240, it is determined whether or not a collation result is consistent. Specifically, in a case where the correlation coefficient calculated in step S230 is equal to or more than a predetermined value, it is determined that a collation result is consistent (Yes), and the flow proceeds to step S250, and, in a case where the correlation coefficient is less than the predetermined value, it is determined that the collation result is not consistent (No), and the flow proceeds to S260. In a case where feature point data of a plurality of users is registered in the registered authentication data 81, the process in steps S230 and S240 are repeatedly performed by changing the feature point data in the registered authentication data 81 for each user.

In step S250, a consistent UID of the registered authentication data 81 is stored in the result data 185. Specifically, a UID having feature point data of which a collation result is consistent in step S240 is acquired from the registered authentication data 81, and the UID, the collation result “1”, the collation time, and the valid flag “1” are stored in the result data 185.

In step S260, the result data 185 is initialized. Specifically, information regarding a UID stored in the result data 185 is erased. Specifically, information regarding a UID is updated to “NULL”, a collation result is changed to “0”, a collation time is changed to “NULL”, and a valid flag is changed to “0”. Since the result data 185 is referred to by the event handler (step S65, and steps S410 to S450) for the payment request event IV5, in a case where a user wearing the wearable apparatus 101 is not registered in advance, card information is guided not to be transmitted through the initialization.

In step S270, the presence or absence of a inheritance is determined, and, in a case where it is determined that there is a inheritance (Yes), the flow proceeds to step S280, and, it is determined that there is no inheritance (No), the flow proceeds to step S290. Regarding the determination of the presence or absence of a inheritance, in a case where the UID acquired in step S250 matches a UID stored in the column 72E of the personal information table 172, it is determined that there is a inheritance, and, in a case where the UID does not match the UID stored in the column 72E, it is determined that there is no inheritance.

In step S280, information regarding a inheritance UID (column 72E) of the personal information table 172 is allowed to be referred to. Specifically, information of the row matching the UID stored in the column 72E of the personal information table 172 in step S270 is read to an internal variable.

In step S285, information regarding the UID of the inheritance source is displayed. Specifically, information related to the UID stored in the column 72A is generated on the basis of the internal variable read in step S280, and is output to the display portion 31. A service or a card number which is taken over may also be displayed.

In step S290, information (non-inheritance information) indicating that there is no inheritance is displayed. For example, the content that “this wearable apparatus is not taken over from any person” is displayed.

Through the flow from steps S270 to S290, in a case where a user wears the wearable apparatus 101, is personally authenticated, and then takes over the right from another user, the content thereof can be checked when wearing the wearable apparatus. For example, when the user U2 wears the wearable apparatus 101, in a case where the right is taken over from the user U1, the content that “this wearable apparatus is taken over from XYZ having the UID “0001” (where XYZ is the name of the user U1) is displayed in the process in step S285.

### (Flow of Unmounting Event Process)

FIG. 10 is a flowchart illustrating a flow of the unmounting event process. This flow corresponds to details of the process in step S50 of the main control process illustrated in FIG. 18.

In step S310 of the present embodiment, data is written into the result data 185. Specifically, in a case where a UID is present (not “NULL”) in the result data 185, the valid flag is changed to “0”. In other words, a result of personal authentication becomes invalid.

The content of the valid flag in the result data 185 is referred to by the event handler for the payment request event IV5, and, in a case where the valid flag is not “1”, card information is not transmitted. In other words, through this step, card information is controlled not to be transmitted in a state in which the wearable apparatus 101 is unmounted.

Also in the present embodiment, it is possible to perform an event process in a case where an unmounting instruction is referred to after the process in FIG. 10 is performed according to the flowchart illustrating a flow of the unmounting event process illustrated in FIG. 12.

Through the flow in FIG. 12, in a case where the unmounting flag is not set to “ON”, and the wearable apparatus 101 is unmounted (detached), the reception processing unit 61 issues an alert sound for prompting a warning.

### (Flow of Authentication Request Process)

FIG. 20 is a flowchart illustrating a flow of the authentication request process. This flow corresponds to details of the process in step S65 of the main control process illustrated in FIG. 18.

In step S410, the result data 185 is read. Specifically, the content of the result data 185 is read to an internal variable. The UID, the collation result, the collation time, and the valid flag are read.

In step S420, it is determined whether or not data is present in the result data 185. Specifically, in a case where the UID is not “NULL”, it is determined that data is present (Yes), the flow proceeds to step S435. In a case where the UID is “NULL”, it is determined that data is not present (No), the flow proceeds to step S450.

In step S435, a UID is acquired for which the collation result is “1”, and the valid flag is “1”. In a case where there is no UID for which both of the collation result and the valid flag are “1”, the flow proceeds to step S450 (not illustrated).

In step S445, card information is transmitted in which the inheritance UID of the personal information table 172 matches the acquired UID. Specifically, information of a row matching the UID stored in the column 72E of the personal information table 172 in step S270 is read to an internal variable. Card information included in the internal variable is transmitted to the charge system 5 via the communication unit 41.

In step S450, a no good (NG) response is transmitted. Specifically, an NG response indicating that card information is not transmitted is transmitted to the charge system 5 via the communication unit 41.

In steps S445 and S450, card information and an NG response are transmitted, but, actually, various pieces of information are transmitted and received in a bidirectional manner on the basis of a communication type, a communication procedure, and a specification of transmission and reception data determined in advance for the wearable apparatus 101 and the charge system 5. Security measures such as encryption are also taken.

### (Flow of Inheritance Event Process)

FIG. 21 is a flowchart illustrating a flow of the inheritance event process. This flow corresponds to details of the process (an event handler for the inheritance event IV7) in step S70 of the main control process illustrated in FIG. 18. The inheritance event IV7 is an event generated by an operation of the user U1 who is a inheritance source.

In step S610, a inheritance destination designation screen is displayed. The screen D10 (FIG. 22) is an example of the inheritance destination designation screen. Information regarding a inheritance UID selected from a selection menu is acquired.

In step S620, a inheritance information designation screen is displayed. The screen D20 (FIG. 23) is an example of the inheritance information designation screen. Inheritance information selected from the selection menu is acquired.

In step S630, the inheritance UID is stored in the personal information table 172. Specifically, a row is selected in which the content of the column 72A of the personal information table 172 matches the UID of the user U1 who is a inheritance source and the contents of the column 72B and the column 72C match the acquired inheritance information, and the information regarding the inheritance UID is stored in the column 72E of the row.

Since the respective flows illustrated in FIGS. 18 to 21 are executed, in a case where a payment process is received from the charge system 5 via the communication unit 41, a notification of the payment request event IV5 is sent, and card information is transmitted through the processes in the authentication request process (step S65 and steps S410 to S450) which is a corresponding event handler. Regarding card information to be transmitted, if the right is set to be taken over from the user U1 who is a inheritance source to the user U2 who is a inheritance destination through the inheritance event process (step S70 and steps S610 to S630), card information of the user U1 is transmitted from the wearable apparatus 101 worn by the user U2. If inheritance setting is not performed through the inheritance event process, card information of the user U2 wearing the wearable apparatus 101 is transmitted. It is possible to transmit card information in a switching manner through the flow of the inheritance event process.

As described above, the wearable apparatus 101 according to the present embodiment can achieve the following effects.

The mounting unit 10 and the detection processing unit 52 detect a mounting state of the wearable apparatus 101. Specifically, in a case where the wearable apparatus 101 is mounted, the mounting event IV1 is output, and, in a case where the wearable apparatus 101 is unmounted, the unmounting event IV3 is output.

If the mounting event IV1 is output, a corresponding event handler calls the authentication processing unit 57, and performs a personal authentication process on the user U1 wearing the wearable apparatus 101. After the user U1 is personally authenticated, a function of the inheritance processing unit 69 can be realized by a predetermined operation of the user U1. The inheritance processing unit 69 sets information to be taken over to a user (user U2) who is a inheritance destination through designation performed by the user U1. In the setting performed by the inheritance processing unit 69, the setting can be performed on a simple setting screen and through a simple operation as illustrated in the screen D10 and the screen D20.

Thereafter, if the wearable apparatus 101 is worn by the user U2, the event handler (authentication processing unit 57) for the mounting event IV1 performs a personal authentication process on the user U2. If the user U2 is personally authenticated, inheritance information (card information or the like of the user U1) which is taken over from the user U1 is displayed on the display portion 31 of the wearable apparatus 101 (step S285). Next, if the card information or the like is requested from the charge system 5, the wearable apparatus 101 worn by the user U2 can transmit the card information of the user U1 by using an event handler for the payment request event IV5.

In the above-described way, the wearable apparatus 101 can safely inherit a result of personal authentication for the user U1 performed by the wearable apparatus 101 to the user U2 according to a simple process.

The present invention is not limited to the above-described embodiments, and various modifications or alterations may be applied to the embodiments. Hereinafter, modification examples will be described by exemplifying the wearable apparatus 1.

### Modification Example 1

In the wearable apparatus 1 according to the embodiment, a case where a payment request is received from the charge system 5 is supposed and exemplified, but an application such as a payment request is only an example. For example, in a viewing system which views asset information or the like by using my number, a user wearing the wearable apparatus 1 comes close to a display screen of the viewing system, and thus asset information or the like of the user can be rapidly displayed. In this case, in a case where a collation result in the result data 85 is “1”, and a valid flag is “1”, the wearable apparatus 1 transmits my number information to the viewing system side. The viewing system can determine a situation in which the my number information is transmitted as a result of the user wearing the wearable apparatus 1 being personally authenticated, and thus displays information cited from my number. By making the wearable apparatus 1 usable in such a viewing system, it is possible to efficiently use the viewing system installed in a public place such as a municipal office.

### Modification Example 2

In the embodiments and the modification example, the authentication processing unit 57 acquires a vein image from the imaging sensor 21 and performs a personal authentication process, but a personal authentication process is not limited to such a method. A personal authentication process may be performed by acquiring, for example, a pulse waveform, an artery image, a blood composition value which can be measured in a noninvasive manner, or a combination thereof.

### Modification Example 3

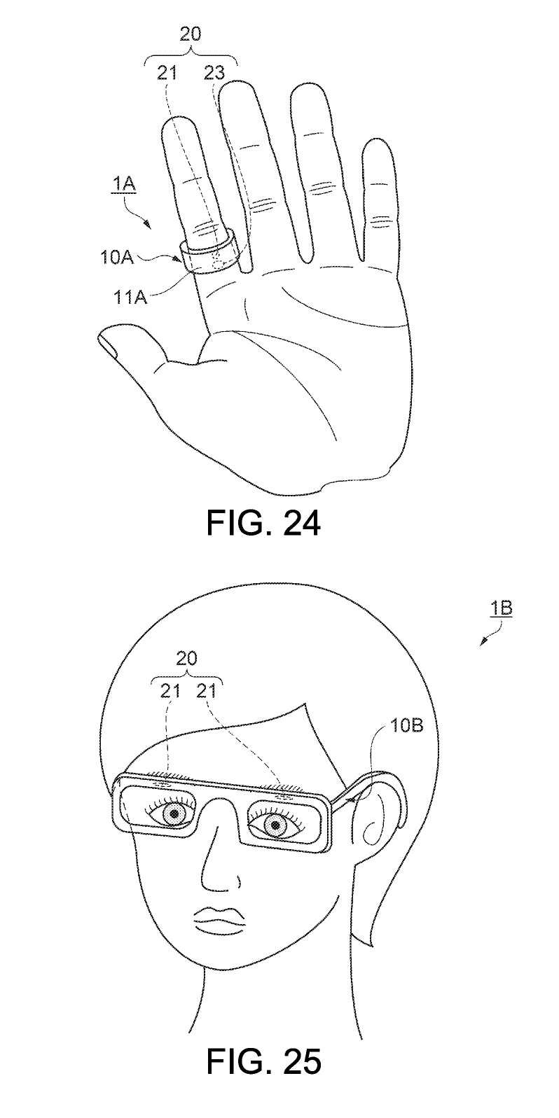

In the embodiments and the modification examples, the wearable apparatus 1 is mounted on the wrist of a user, but the wearable apparatus 1 may be an apparatus mounted on a part other than the wrist. Modification examples thereof will be described with reference to the drawings. FIGS. 24 to 28 are explanatory diagrams illustrating an overview of a usage form of a wearable apparatus according to a modification example.

### (1) Ring Type or Fingerstall Type Wearable Apparatus

A wearable apparatus of the present modification example may be a ring type or fingerstall type wearable apparatus mounted by wrapping a band of a mounting unit around a finger or a toe. For example, a ring type wearable apparatus 1A illustrated in FIG. 24 is mounted by wrapping a band 11A of a mounting unit 10A around the finger. Then, the biological information measurement unit 20 acquires a blood vessel image of the finger by using the imaging sensor 21, and performs a personal authentication process on the basis of a blood vessel pattern. A pulse waveform may be used for a personal authentication process by using the biological sensor 23. A fingerprint image of a finger or a wrinkle image of a finger pad is acquired, and a personal authentication process is performed by a fingerprint pattern or a wrinkle pattern. A valid flag is set to “1” after a user is personally authenticated until the band 11A is removed.

### (2) Spectacle Type Wearable Apparatus

A wearable apparatus of the present modification example may be a spectacle type wearable apparatus of which a mounting unit is mounted on the face as frames of spectacles. For example, in a spectacle type wearable apparatus 1B illustrated in FIG. 25, a mounting unit 10B is mounted on the face. Then, the biological information measurement unit 20 captures images of the pupils by using the imaging sensors 21, acquires capillary images of the retinae or iris images, and performs a personal authentication process by using a capillary pattern or an iris pattern. A valid flag is set to “1” after a user is personally authenticated until the frames which are the mounting unit 10B are removed from the face.

### (3) Glove Type Wearable Apparatus

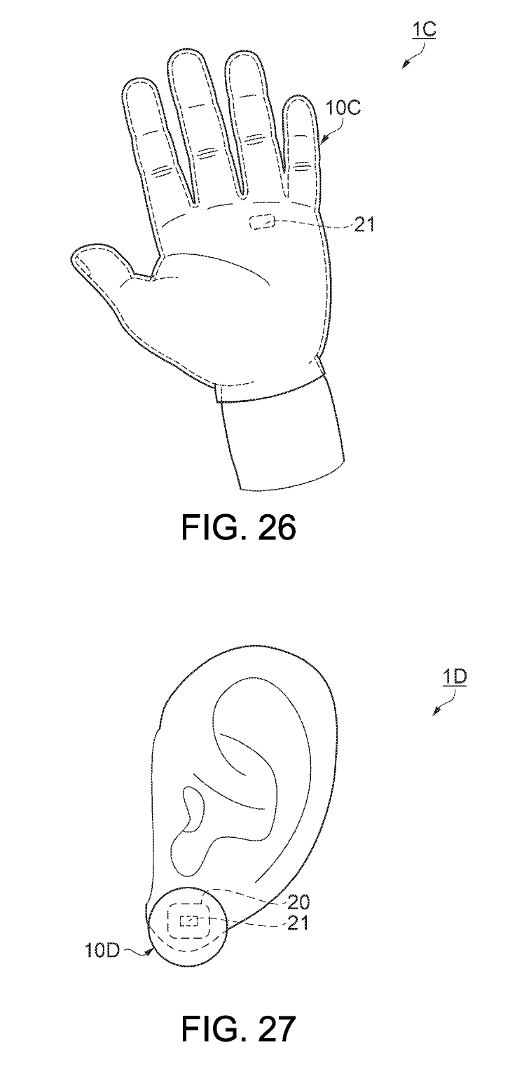

A wearable apparatus of the present modification example may be a glove type or palm mounted type wearable apparatus of which a mounting unit is mounted on the hand or the palm. For example, in a glove type wearable apparatus 1C illustrated in FIG. 26, if a mounting unit 10C is mounted on the hand, the biological information measurement unit 20 acquires a palm image or a vein image obtained by imaging wrinkles of the palm by using the imaging sensor 21, and performs a personal authentication process by using a wrinkle pattern or a vein pattern of the palm. A valid flag is set to “1” after a user is personally authenticated until the mounting unit 10C is removed from the hand.

### (4) Ear Mounted Type Wearable Apparatus

A wearable apparatus of the present modification example may be an ear mounted type wearable apparatus having a shape of a headphone, an earphone, or an earring, of which a mounting unit is mounted on the ear. For example, in an earring-shaped wearable apparatus 1D illustrated in FIG. 27, if a mounting unit 10D is mounted on the ear, the biological information measurement unit 20 captures an ear shape image or a vein image of the ear lobe by using the imaging sensor 21, and performs a personal authentication process by using an ear shape pattern or a vein pattern. A valid flag is set to “1” after a user is personally authenticated until the mounting unit 10D is removed from the ear.

### (5) Clothing Type Wearable Apparatus

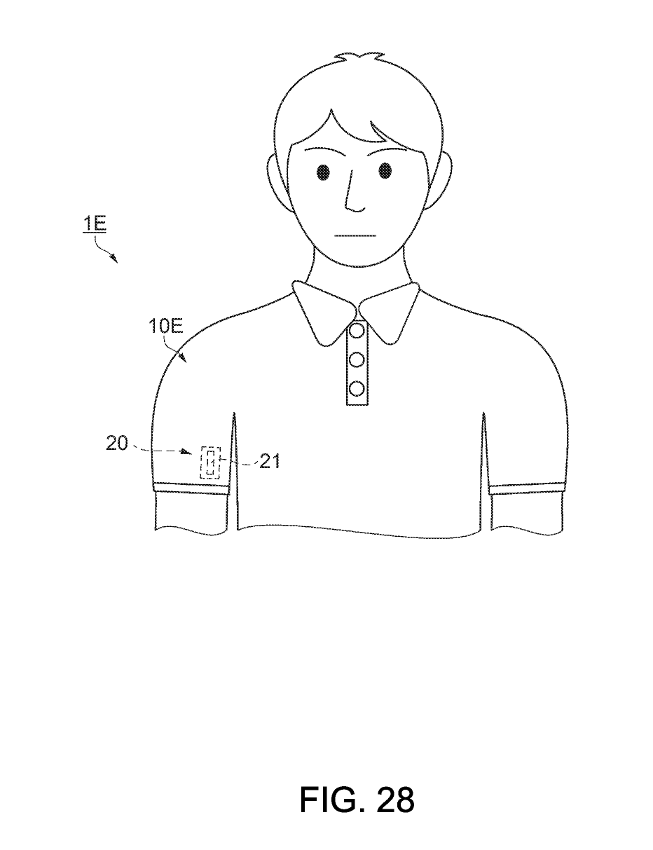

A wearable apparatus of the present modification example may be a clothing type wearable apparatus having a shape of a shirt, a sweater, pants, or socks, of which a mounting unit is mounted on the body. For example, in a shirt-shaped wearable apparatus 1E illustrated in FIG. 28, if a mounting unit 10E is mounted on the body, the biological information measurement unit 20 captures and acquires a vein image of any part of the body or a physical feature image (a position or a size of a mole) by using the imaging sensor 21. A personal authentication process is performed by using a vein pattern or a physical feature pattern. A valid flag is set to “1” after a user is personally authenticated until the wearable apparatus 1E is taken off.

### Modification Example 4

In the embodiments and the modification examples, the mounting unit 10 monitors an ON/OFF signal from the opening/closing portion 12, and the detection processing unit 52 detects mounting and unmounting, but this configuration is only an example. For example, the band 11 of the mounting unit 10 is a metallic bracelet annularly mounted on the wrist of a user. The mounting unit 10 is provided with an adjustment portion, and the adjustment portion is a band pressing member which can variably adjust a circumferential length of an annular shape of the band 11. The band pressing member may be a one-side opening type buckle, a both-hole type buckle, or a double lock type buckle. The adjustment portion is provided with a mechanism such as a rotary mechanism measuring a circumferential length including a circumferential length of the band 11 fixed to the wrist, an optical sensor which counts a constant length, or a switch sensor which detects bending of the buckle, and outputs a measured circumferential length.

It is possible for the detection processing unit 52 to increase detectability of mounting and unmounting of a wearable apparatus in a user by using such a mechanism.

Specifically, when a user mounts the wearable apparatus 1 on the wrist, the initial registration unit 65 performs a personal authentication process. In this case, the initial registration unit 65 measures a circumferential length (a circumferential length in a mounting state) when the user is personally authenticated. The measured circumferential length in a mounting state is stored in the storage unit 70.

The detection processing unit 52 (mounting detection portion 53) acquires a circumferential length of the band 11 from the adjustment portion, detects that the wearable apparatus 1 is mounted on the wrist of the user in a case where the circumferential length is equivalent to the circumferential length in a mounting state stored in the storage unit 70, and outputs the mounting event IV1. In a determination of the circumferential length being equivalent, in a case where the circumferential length of the band 11 is smaller than the circumferential length in a mounting state by a length obtained by adding a predetermined length (for example, 2 mm) thereto in consideration of an error during mounting.

Thereafter, in a case where a measured circumferential length of the band 11 is equal to or more than the circumferential length in a mounting state by a predetermined length (for example, 2 mm), the detection processing unit 52 (unmounting detection portion 55) detects that the wearable apparatus 1 is detached from the wrist of the user, and outputs the unmounting event IV3.

In a case where pressure data (contact pressing) output from a pressing sensor provided in the opening/closing portion 12 is equal to or more than predetermined pressure, the mounting event IV1 may be output. As the predetermined pressure, pressure data acquired through a process in the initial registration unit 65 of the control unit 50 when a user wears the wearable apparatus 1 is registered. The predetermined pressure may be acquired as differing pressure data depending on the circumference of the wrist of a user or elasticity of the wrist.

With the above-described configuration, it is possible to detect whether or not a user wearing the wearable apparatus 1 is personally authenticated therein, and thus to realize a more reliable personal authentication process.

### Modification Example 5

In the embodiments and the modification examples, the control unit 50 sets a valid flag of the result data 85 to “0” through a process in an event handler for the unmounting event IV3, but is not limited to such a configuration. Setting of the valid flag of the result data 85 to “0” may be performed according to timings of the following configurations (1) to (3).

The determination processing unit 59 may output a determination result by referring to the set valid flag at these timings. The authentication processing unit 57 may be called to perform personal authentication at these timings. Each of the configurations (1) to (3) may be used alone, and may be obtained by a plurality of configurations.

### (1) Biological Maintenance Information Acquisition Unit (Change in Biological Maintenance Information)

The wearable apparatus 1 may further include a biological maintenance information acquisition unit which acquires biological maintenance information for determining that a biological state of a user is maintained, and may set the valid flag to “0” in a case where there is a change in acquired biological maintenance information. The biological maintenance information is an example of biological reaction information, and the biological maintenance information acquisition unit corresponds to a biological reaction information acquisition unit.

The biological maintenance information is information regarding a biological reaction such as a pulse waveform, a pulse rate, a skin temperature, or the temperature of a skin deep part, measured by the biological sensor 23. The biological maintenance information is continuously measured every predetermined period (for example, once/second). A biological maintenance state is a state of maintaining survival, maintaining consciousness, or maintaining awakening, and, in a case where each state changes and thus a biological maintenance state changes, for example, survival is interrupted, consciousness is lost, or a user falls asleep, a measured value of the biological maintenance information changes. A situation in which a measured value changes is monitored and is compared with a predetermined state, and can thus it is possible to recognize a change in the biological maintenance state. For example, in a case where a predetermined pulsation waveform is not present in a pulse waveform, it is determined that the biological maintenance state has changed.

As the biological maintenance information, information such as no shaking, no sudden change in momentum, or no sudden change in motion intensity is calculated. This information may be calculated on the basis of a relationship between body motion information per unit time acquired from an acceleration sensor provided in the operation unit 37 and a change in a pulse rate.

In a case where such a change situation reaches a predetermined state, an event is generated, and an event handler for the event sets the valid flag of the result data 85 to “0”.

With the above-described configuration, in a case where a user loses payment capability or payment determination capability, for example, survival of the user is interrupted, the consciousness of the user is lost, or the user falls asleep, coping of not receiving a payment process is possible. In a case where it is estimated that a user has some sort of high-risk stimulus from the outside, for example, the user is shaking, a sudden action is given to the user, or sudden loading motion is performed by the user, coping of not receiving a payment process is possible.

### (2) Clocking Unit (Change in Time Point)

In a case where a time point acquired from the clocking unit 39 reaches a predetermined time point, the wearable apparatus 1 sets the valid flag to “0”.

The predetermined time point is set for a user by using the initial registration unit 65 of the control unit 50, and a time point until a result of personal authentication is valid is set. In other words, a result of a personal authentication process is valid up to the predetermined time point, but, if the predetermined time elapses, the result of the personal authentication process is invalid. The predetermined time point is set to, for example, 6:00 every morning, and thus the result of the personal authentication process becomes invalid at 6:00 every morning, and, in a case where the wearable apparatus 1 is desired to be continuously used, a personal authentication process may be performed again. The predetermined time point may be an elapsed time.

With this configuration, since a personal authentication process is performed every predetermined time point, the robustness of security is increased, and a predetermined time from the predetermined time point (for example, for thirty minutes from 6:00 every morning) can be intentionally secured as the time required for a personal authentication process.

### (3) Position Information Acquisition Unit (Change in Position Information)

The wearable apparatus 1 may further include a positioning sensor (corresponding a position information acquisition unit) acquiring position information of a user, and may set the valid flag to “0” in a case where position information acquired from the positioning sensor is deviated from a predetermined range.

The position information is position information calculated on the basis of a signal acquired from, for example, a global navigation satellite system (GNSS) satellite, and may be continuously acquired every predetermined time (for example, once/second).

The predetermined range is a range set for a user by using the initial registration unit 65 of the control unit 50, and is set to a behavior range or the like of the user including a location where the user may perform a payment process. In other words, in a case where the wearable apparatus 1 is deviated from the behavior range of the user, the valid flag is set to “0”, and thus the robustness of security can be further increased in a location where a payment process is not required to be performed.

### Modification Example 6

In the embodiments and the modification examples, there may be provided a temporary reception unit which allows a user who is personally authenticated to temporarily unmount a wearable apparatus during mounting through an operation of the user.

The temporary reception unit controls the operation unit 37 and the display portion 31, and acquires a simple collation key from the user. The simple collation key is, for example, characters of four digits or an answer to a question. The temporary reception unit outputs the simple collation key to the authentication processing unit 57.

If the simple collation key is input from the temporary reception unit, the authentication processing unit 57 prompts the user to input a simple collation key when called by an event handler for the mounting event IV1. In a case where the character string which is input by the user matches the simple collation key, it is determined that the user is personally authenticated, the collation result of the result data 85 is set to “1”, and the valid flag is set to “1”. In a case where the simple collation key is not consistent, a regular personal authentication process is performed.

With this configuration, for example, in a case where a user desires to temporarily unmount a wearable apparatus according to the user's intention, the user can return to personal authentication in a simple procedure. For example, this function is convenient before and after bathing or before and after hand washing. Collating the simple collation key corresponds to performing simple personal authentication.

### Modification Example 7

In the embodiments and the modification examples, in the reception processing unit 61, a predetermined operation performed by a user is to input a simple password for collation, but the predetermined operation is not limited to an input operation. For example, the predetermined operation may be an action operation such as an operation of swinging the wearable apparatus 1 or an operation of tapping the wearable apparatus 1. The predetermined operation may be specified by comparing a pattern of an acceleration signal detected by the acceleration sensor provided in the operation unit 37 with a predetermined pattern.

In the temporary reception unit in the above-described modification example, input of a collation key and collation have been exemplified, but an action operation on the wearable apparatus 1 may also be employed.

### Modification Example 8

In the embodiments and the modification examples, the authentication request process (step S60) illustrated in FIG. 7 is an event handler in a case where the payment request event IV5 occurs, but a process of determining in advance whether or not a payment request is received from a payment process system may be added thereto. For example, in a case where a user performs a predetermined operation (for example, tapping the wearable apparatus 1 three times) before a payment process is performed, a process of receiving a payment request is added. By adding such a predetermined operation, the communication unit 41 and the request detection processing unit 56 can be brought into a reception standby state only when a payment process is necessary, and thus it is possible to reduce power consumption required for reception standby when a payment process is not necessary.

### REFERENCE SIGNS LIST

1 WEARABLE APPARATUS
5 CHARGE SYSTEM
5A PAYMENT PROCESS
5B PAYMENT PROCESS
7 COMMUNICATION
10 MOUNTING UNIT
11 BAND
12 OPENING/CLOSING PORTION
20 BIOLOGICAL INFORMATION MEASUREMENT UNIT
21 IMAGING SENSOR
23 BIOLOGICAL SENSOR
30 NOTIFICATION UNIT
31 DISPLAY PORTION
33 SOUND OUTPUT PORTION
35 VIBRATION PORTION
37 OPERATION UNIT
39 CLOCKING UNIT
41 COMMUNICATION UNIT
50 CONTROL UNIT
52 DETECTION PROCESSING UNIT
53 MOUNTING DETECTION PORTION
55 UNMOUNTING DETECTION PORTION
56 REQUEST DETECTION PROCESSING UNIT
57 AUTHENTICATION PROCESSING UNIT
59 DETERMINATION PROCESSING UNIT
61 RECEPTION PROCESSING UNIT
63 NOTIFICATION PROCESSING UNIT
65 INITIAL REGISTRATION UNIT
67 EVENT PROCESSING UNIT
70 STORAGE UNIT
72 PERSONAL INFORMATION TABLE
73 PERSONAL INFORMATION DATA
75 CARD INFORMATION DATA
81 REGISTERED AUTHENTICATION DATA
83 ACQUIRED AUTHENTICATION DATA
85 RESULT DATA

1. A wearable apparatus, configured to be worn by a user, and comprising:
an authentication unit that performs personal authentication for the user wearing the wearable apparatus;
a detection unit that detects a mounting state of the wearable apparatus;
a determination unit that determines whether a result of the personal authentication is valid or invalid on the basis of the mounting state detected by the detection unit; and
a control unit that omits the personal authentication in the authentication unit, and outputs a result of the personal authentication, in a case where the user is personally authenticated by the authentication unit, and then the result of the personal authentication is determined as being valid by the determination unit.

2. The wearable apparatus according to claim 1, further comprising:
a reception unit that receives an unmounting instruction for the wearable apparatus; and
a notification unit that performs a notification of information by stimulating senses of a living body including at least one of a vision, an auditory sense, and a tactile sense,
wherein the control unit causes the notification unit to perform a notification of abnormality information in a case where it is detected by the detection unit that the wearable apparatus is unmounted during non-reception in which the user does not give the unmounting instruction to the reception unit.

3. The wearable apparatus according to claim 1, further comprising:
a storage unit that stores financial process information which enables a financial process using at least one of a credit card function, a cash card function, a debit card function, and a net banking function to be performed,
wherein the control unit performs the financial process in a case where the user wearing the wearable apparatus is personally authenticated by the authentication unit, and then the result of the personal authentication is determined as being valid by the determination unit, and
wherein the control unit causes the financial process not to be performed in a case where the result of the personal authentication is determined as being invalid.

4. The wearable apparatus according to claim 1, further comprising:
a mounting unit that is used for the wearable apparatus to be worn by the user,
wherein the mounting unit forms an annular shape around the wrist of the user,
wherein the mounting unit has an opening/closing portion which opens and closes the annular shape, and
wherein, in a case where the opening/closing portion is opened, the detection unit detects that the wearable apparatus is unmounted.

5. The wearable apparatus according to claim 1, further comprising:
a mounting unit that is used for the wearable apparatus to be worn by the user,
wherein the mounting unit forms an annular shape around the wrist of the user,
wherein the mounting unit has an adjustment portion which variably adjusts a circumferential length of the annular shape, and
wherein, in a case where a circumferential length in a mounting state is increased by a predetermined length by the adjustment portion, the detection unit detects that the wearable apparatus is unmounted.

6. The wearable apparatus according to claim 1,
wherein the authentication unit acquires biological information of the user, and performs the personal authentication.

7. The wearable apparatus according to claim 1,
wherein the authentication unit performs the personal authentication by using a password entered by the user.

8. The wearable apparatus according to claim 1,
wherein the authentication unit is connected to an authentication server via a computer communication network, and performs the personal authentication by collating information acquired from the user with specific information of the user stored in the authentication server.

9. The wearable apparatus according to claim 1, further comprising:
a biological reaction information acquisition unit that acquires biological reaction information by detecting a biological reaction of the user,
wherein the determination unit determines whether the result of the personal authentication is valid or invalid on the basis of information regarding whether or not the biological reaction information is continuously measured by the biological reaction information acquisition unit.

10. The wearable apparatus according to claim 1, further comprising:
at least one of a clocking unit that measures a time point, a position information acquisition unit that acquires a position of the wearable apparatus, and a biological reaction information acquisition unit that acquires biological reaction information by detecting a biological reaction of the user,
wherein, in at least one of a case where the time point becomes a predetermined time point, a case where the position is deviated from a predetermined range, and a case where the biological reaction information changes, the authentication unit performs the personal authentication for the user.

11. The wearable apparatus according to claim 1, further comprising:
a temporary reception unit that receives temporary unmounting of the wearable apparatus,
wherein, in a case where the temporary unmounting is received from the personally authenticated user, the control unit causes the authentication unit to perform simple personal authentication.

12. The wearable apparatus according to claim 1,
wherein the detection unit measures contact pressing against the wrist of the user, and detects the mounting state on the basis of the contact pressing of when the user wears the wearable apparatus.

13. The wearable apparatus according to claim 1, further comprising:
a power generation unit that generates power,
wherein the wearable apparatus is operated by using power from the power generation unit.
